(19) United States
(12) Patent Application Publication
(10) Pub. No.: US 2010/0255059 A1
(43) Pub. Date: Oct. 7, 2010

(54) Artificial micro-gland

(75) Inventors: Manuel Marquez, Midlothian, VA (US); Samantha M. Marquez, Midlothian, VA (US); Antonio Garcia, Chandler, AZ (US)

Correspondence Address: LOUIS VENTRE, JR, 2483 OAKTON HILLS DRIVE, OAKTON, VA, 22124-1530, US

(73) Assignee: YNANO, LLC, Midlothian, VA (US)

(21) Appl. No.: 12/726,158

(22) Filed: Mar. 17, 2010

Related U.S. Application Data
(60) Provisional application No. 61/257,666, filed on Nov. 3, 2009.
(60) Provisional application No. 61/165,989, filed on Apr. 2, 2009.

(51) Int. Cl. A61K 35/14; A61K 9/00; A61K 35/16; A61K 35/18; A61K 35/12; A61P 5/00; C12N 5/07; C12N 5/0789; C12N 5/073; C12N 5/09; C12N 5/078

(52) U.S. Cl. 424/424; 424/93.71; 424/93.72; 424/93.73; 424/93.7; 435/325; 977/894

(57) ABSTRACT

A micro-scale artificial gland is disclosed in the form of an independent unit for promoting biological activity. The artificial gland includes cells formed in a membrane enclosing a reservoir. The reservoir is a bio-reactor capable of containing a product of activity of the cells. The reservoir comprises a gas, a liquid, and a gel and preferably also contains nanoparticles, a buffer, a surfactant, and, a gel precursor. The reservoir may also contain cells. Nanoparticles may also surround the artificial gland to form a protective coating. A variety of methods are disclosed for making the artificial gland by directed assembly of cells into the artificial micro-gland by gel, liquid or bubble templating. All involve coating the surface of gel, droplet or bubble with the living cells and the stabilizing the cells on the surface of gels, droplets or bubbles.

### CROSS-REFERENCE TO RELATED APPLICATIONS

This application claims the benefit of U.S. Provisional Application No. 61/257,666, filed 3 Nov. 2009, and U.S. Provisional Application No. 61/165,989 filed 2 Apr. 2009, which are hereby incorporated by reference herein.

### TECHNICAL FIELD

In the field of bio-affecting and body-treating compositions, an artificial gland of micro-scale with a cellular membrane and bioreactor reservoir, wherein the artificial gland is useful for biological tissue and organ repair and replacement and stem cell engineering and biotechnology applications.

### BACKGROUND ART

Tissue and organ engineering are popular terms used to describe efforts to form complex living structures using cells as building blocks. However, no man-made method has yet been described that will permit growth of complex organs. The artificial gland of the present invention can be used to enable the growth of complex organs.

The term “tissue engineering” is commonly used to describe the range of techniques involved in forming simple, essentially 2-dimensional arrays of cells that work in concert to generate tissue-like function and to describe the manufacturing of entire, simple organs.

By familiarity and necessity, the method used to organize cells into living tissue was initially based on standard microbiological methods using Petri dishes and shallow cell culture bottles in order to create a confluent surface of cells.

More sophisticated tissue structures are presently possible using scaffolding, which requires the use of a macro-scale material that can promote 3-dimensional cell organization into tissue by providing a surface for cell attachment and proliferation. Scaffolding materials are designed to eventually become engulfed by the tissue or, more acceptably, be slowly removed by natural degradation or dissolution in the body.

The present invention provides a tiny artificial gland in the size range of millimeters to micrometers that eliminates the need for a macro-scale tissue-shaping scaffold.

Tissue engineering using the artificial gland of the present invention solves a host problems resulting from the use of macro-scale scaffolds, such as inflammatory response, release of potentially toxic substances, and differences in tissue function when the tissue is created from a scaffold.

Given the need to organize cells in structures other than sheets or mats, prior art teachings were logically based on the assumption that a physical material, that is a macro-scale scaffold, would be needed to force cells into shapes.

While nature does not require preformed structures, the time scale for producing complex organisms (e.g., the gestation period) can be relatively long with respect to the timeline for most patient care. In nature, the instructions for forming each and every tissue and organ are predetermined and come about from a single cell or from the fusion of two cells, since this is the starting point for all multicellular organism growth and development. However, no man-made process that mimics nature is taught in the prior art.

### SUMMARY OF INVENTION

An artificial gland is disclosed in the form of an independent unit for promoting biological activity. It is a “living capsule” with a biomembrane (tissue) shell and a unique core that acts as container or reservoir. The artificial gland is preferably of micro-scale. It includes cells formed in a membrane enclosing a reservoir. The reservoir is a bio-reactor capable of containing a product of activity of the cells. The reservoir preferably comprises a gas, a liquid, or a gel and preferably also contains nanoparticles, a buffer, a surfactant, and, a gel precursor. The reservoir may also contain cells. Nanoparticles may also surround the artificial gland to form a protective coating.

A variety of methods of making the artificial gland are disclosed. These include strategies to encourage cell formation on the surface of a gel, gas bubble or liquid droplet using nanoparticles that cross-link to the cells. These processes drive and organize living cells (yeast, fibroblast, etc) to the surface of a gel, liquid or gas (bubble) by controlling the cells and templates surfaces using LbL polyelectrolyte decoration, selective gelation using CaCO3 nanoparticles-cell composites, and, hydrophobic deposition.

### Technical Problem

The prior art describes no artificial glands that can be employed for tissue or organ engineering without using macro-scale scaffolds. More specifically, there are no artificial glands with a membrane of cells and a central reservoir that: mimic nature; create opportunities to trigger events that can lead to complex tissues, organs, organisms, and vehicles for food and pharmaceutical applications; and, model and control stem cell fate: stem cell behavior and cellular differentiation.

The current state-of-the-art does not allow for the preparation of an artificial gland structure having a heterogeneous cell membrane composition surrounding a reservoir.

The current state-of-the-art does not allow for flexibility in the level of design and control needed to work with different types of cells, biological units and components of cells, which is needed for the creation of heterogeneous or complex morphologies.

The current state-of-the-art does not allow for control over the proper arrangement of multiple types of cellular and subcellular units in three dimensions.

Researchers currently have limited ability to mimic the natural stem-cell micro-environment. The two most popular methods that employ matrices to attempt the 3-D cell culture especially focused on stem cells are: a) the use of shrink-dink hanging drops; and, b) the use of micro-molded micro wells to guide the spontaneous self-assembly of cells into 3-D micro tissues. Both methods are tedious, multiple-step processes, with major inconveniences and design limitations inherent in the processes of guiding cells self-assembly of cells into 3-D micro-tissues with no control or versatility in size, shapes, and structures that limit the ability to mimic the natural stem cell microenvironment.

### Solution to Problem

The solution is a micrometer-to-millimeter-scale artificial gland comprising a membrane of cellular material surrounding a reservoir comprising a bioreactor. The artificial gland is capable of being used to support the growth of organs and other biological material without the use of macro-scale scaffolds. The artificial gland can control the 3-dimensional arrangements of cells and subcellular systems in such a way that can mimic nature.

The solution is a tiny artificial gland that uses cells, biological units, or cellular subunits as the membrane of an artificial gland.

The solution is a method for organizing or growing functional living tissue and complex structures from the artificial gland.

### ADVANTAGEOUS EFFECTS OF INVENTION

The artificial gland of this invention can be used accomplish precise control of the spatial arrangement of cells as well as segregation and assembly of different types of cells. It holds the potential to play a vital role in tissue engineering, stem cell engineering, synthetic biology, and in the design of multicellular vehicles for food and pharmaceutical applications.

New biological and pseudo-biological organization of subunits enables unique structures for the design and control of biological activity in 3-dimensional space.

The invention provides a new ability to control and arrange subcellular and cell-like structures, such as vesicles and liposomes, in 3-dimensional structures for the packaging and transport of biologicals.

The invention provides an artificial means to arrange cells, biological units and subcellular structures similarly to natural multicellular organism development, while adding capability to control spatial location and confinement through the use of external fields, microfluidic channels, and solvent-phase partitioning.

The invention provides new means for manipulating controlled releases or absorptions supporting biological activity. In addition to tissue engineering, this new means is applicable to tuning rheological or optical properties of cosmetics, foods, or other fluids. Sections of the artificial gland can be functionalized for a specific biological tasking.

The invention discloses for the first time a new process of making unique and complex artificial glands with a cellular or biological unit membrane preferably in a micrometer size range.

The artificial gland of the invention will serve as a tool for the future design and control of stem cell fate: stem cell behavior and cellular differentiation.

The invention has application to three-dimensional (3-D) in vitro cell cultures, in which cells are grown in environments that more closely mimic native tissue architecture and function. These applications are important in developmental/cell biology and regenerative medicine. The present invention solves nagging problems inherent in 3-D cell cultures by providing a uniquely configurable core/shell living micro-capsule or artificial micro-gland, which delivers a needed ability to control cell architecture in the shell while maintaining the core as an artificial micro-environment. The artificial micro-gland is model that serves as a tool for the future design and control of the stem cell fate: stem cell behavior and cellular differentiation.

### BRIEF DESCRIPTION OF DRAWINGS

The drawings illustrate preferred embodiments of the method of the invention and the reference numbers in the drawings are used consistently throughout. New reference numbers in FIG. 2 are given the 200 series numbers. Similarly, new reference numbers in each succeeding drawing are given a corresponding series number beginning with the figure number.

FIG. 1 illustrates artificial glands types and a precursor particle.

FIG. 2 is a flow diagram illustrating electrocoalescence of two particle-stabilized droplets to make an artificial gland of a Janus-type.

FIG. 3 is a flow diagram illustrating directionality-flow of artificial glands coalesced to form a complex membrane.

FIG. 4 is a flow diagram illustrating microfluidic formation of an artificial gland.

FIG. 5 is a flow diagram illustrating formation of a colloidosome with different particles and liquids, also known as a double Janus structure.

FIG. 6 is an illustration of motifs for arranging artificial glands.

FIG. 7 includes micrographs illustrating formation of an artificial glands.

FIG. 8 is a flow diagram illustrating formation of artificial glands production.

FIG. 9 illustrates four potential methods for organizing artificial glands.

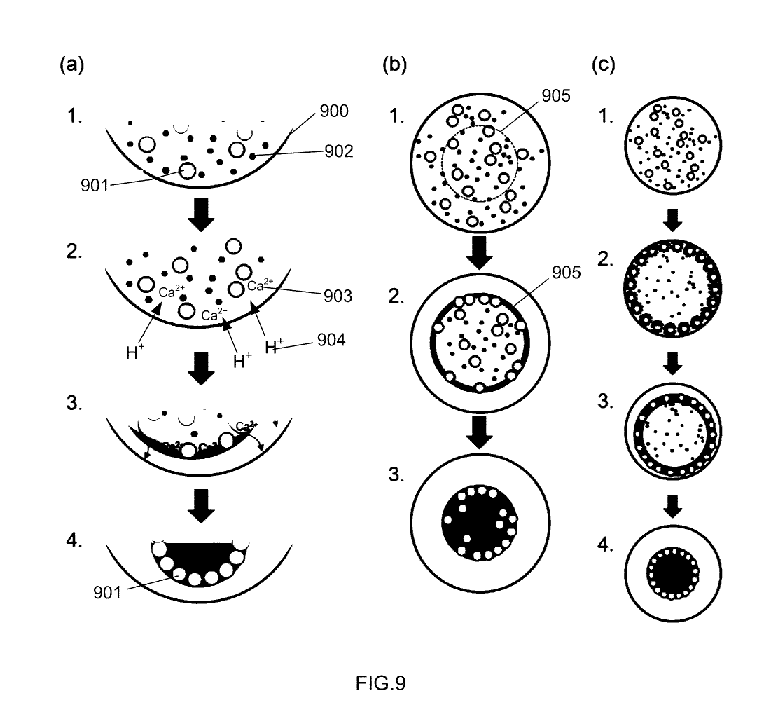

FIG. 10 is a flow diagram illustrating formation of Janus-type artificial glands.

FIG. 11 is an illustration of an artificial gland with membrane-reservoir structure in which islet cells are contained within the reservoir.

FIG. 12 is a flow diagram illustrating formation of artificial gland-based glands for insulin delivery/release applications.

### DESCRIPTION OF EMBODIMENTS

In the following description, reference is made to the accompanying drawings, which form a part hereof and which illustrate several embodiments of the present invention. The drawings and the preferred embodiments of the invention are presented with the understanding that the present invention is susceptible of embodiments in many different forms and, therefore, other embodiments may be utilized and structural, and operational changes may be made, without departing from the scope of the present invention. For example, the steps in the method of the invention may be performed in any order that results making or using the artificial gland.

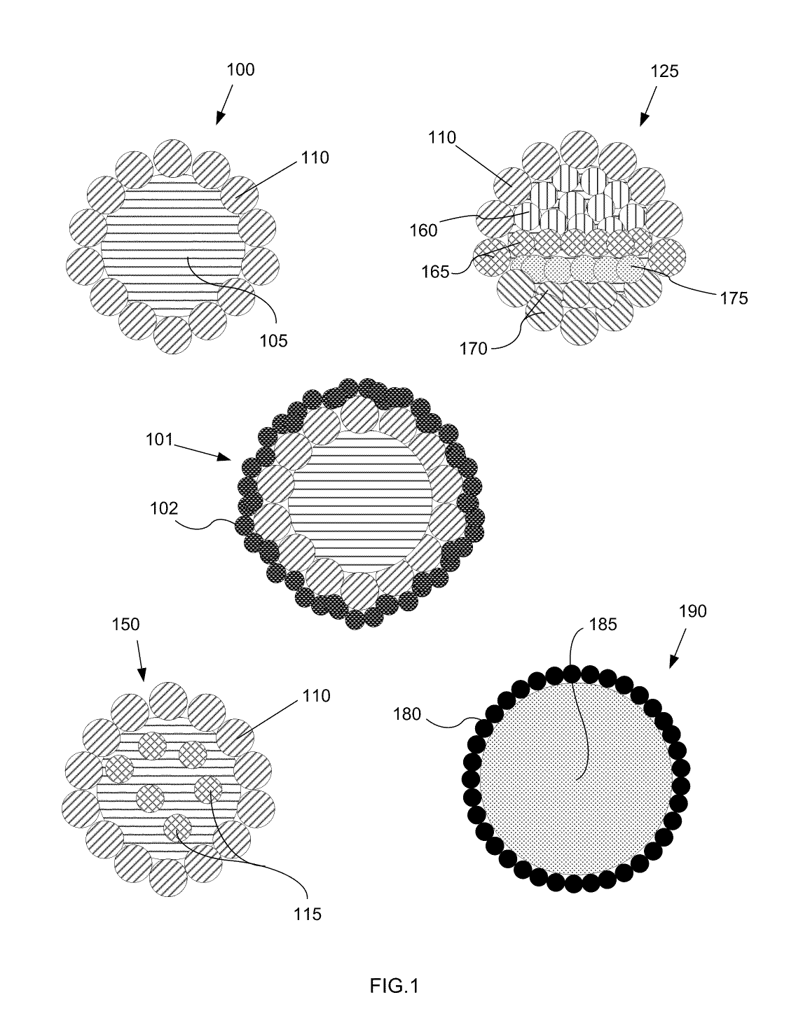

FIG. 1 shows three preferred embodiments of the artificial gland of the invention: a first artificial gland embodiment (100); a second first artificial gland embodiment (125); and a third first artificial gland embodiment (150). Each such embodiment is discussed below.

In its simplest form, the first artificial gland embodiment (100) is essentially first cells (110) surrounding a first reservoir (105) and is an independent micro-scale unit for promoting biological activity.

For all of the embodiments, the artificial gland, as an independent unit, is an isolated product that can be assembled into tissue, organs, or other biological supportive material. Preferably, the artificial gland is in the micron size range of about 10-500 microns. However, larger embodiments up to a centimeter and beyond in diameter are theoretically possible.

The term “cells,” as used herein for all of the embodiments, refers to the structural and functional unit of all known living organisms. In this sense, the cell is itself living and functions to produce chemicals, proteins or other products supporting biological activity. As used herein, each cell is a living structural unit with an individual size in the range of microns to millimeters.

The first artificial gland embodiment (100) comprises first cells (110) assembled in three dimensions and organized to form a membrane. A plurality of cells, thus, forms a membrane. The membrane is configured to define or enclose a closed micro-scale volume. The shape of this configuration may be spherical, spheroidal, discoid, cylindrical, tubular or any other three-dimensional shape that physically defines an internal micro-scale volume. The cells may be of a single type as shown for the first cells (110) of the first artificial gland embodiment (100), or may be multiple or mixed types of cells (160, 165, 170, 175), as shown in the second artificial gland embodiment (125). In FIG. 1 and the other figures, different shading in the cells is intended to reflect different cell types.

The first artificial gland embodiment (100) next comprises a reservoir, shown in FIG. 1 as first reservoir (105), within the enclosed micro-scale volume. The reservoir comprises and essentially is a bio-reactor that supports a biologically active environment and is capable of containing a product of activity of the cells, for example the first cells (110) shown in FIG. 1, in the membrane. The contents of the bio-reactor preferably include a substance comprising a fluid in the form of a gas, liquid, gel, or a combination of these. A fluidic substance has a tendency to assume the shape of the micro-scale volume. The reservoir may also contain other components, such as cells (115) as shown in the third artificial gland embodiment (150); and a plurality of different types of cells (160, 165, 170 and 175), as shown in the second artificial gland embodiment (125).

In alternative embodiments, the artificial gland may be in the form of a tubular, or cylindrical, fiber either closed at both ends, or joined at both ends in a toroidal shape.

In the topological control of cell-shell capsules, diffusive oxygen transport into cell aggregates is one of the major limiting factors of tissue engineering, and the practical size limit for spherical aggregates of cells, not in an artificial gland structure, has been found to be approximately 100-200 micrometers.

The artificial gland structure can overcome this size limit in all dimensions other than cross-section. The artificial gland's versatility in structural shape greatly expands the potential applications. Living cells or tissue membranes surrounding a reservoir having a variety of shape comprising a sphere, a cylinder, a toroid, and any other shapes are within the scope of the invention.

It is expected that, in cross section, any shell, cylinder or toroid will have the same size-limit of approximately 100-200 micrometers, yet there is absolutely no fundamental limitation on any of the other dimensions of these shapes. Cylinders can be any length, toroids can have any major radius, shells of any shape can have any size. Shape variability dramatically broadens the parameter-space for the design of any type of artificial tissue, and can help to direct strategies for all types of tissue engineering.

It is also noted that artificial glands can be assembled in any combination including those where one artificial gland is within another artificial gland. This sort of combination is envisioned where multiple cell growth functions would be helpful to tissue or organ regeneration.

This method provides great flexibility in tuning the aspect ratio of the Toroidal Celloidosome.

The invention is functional with any type of cells. Examples of cells that may be used in the various embodiments are stem cells, mesenchymal cells, embryonic cells, hybridomes B, hybridomes T, differentiated cells, tumor cells, cancer cells, skin cells, neural tube cell derivatives, astrocytes, olygodendrocytes, neuron, muscle cells, myocytes, myocardiocytes, leiomyocytes, epithelial cells, endothelium cells, endocrine gland cells, immune system cells, phagocytes, macrophages, lymphocytes, white cells, thrombocytes, platelets, erythrocytes, red cells, neutrophils, mastocytes, eosinophils, hematopoietic precursor cells, cells from a erytocyte line, proerytroblast, erythroblast basophil, erythroblast polychromatophilo, erythroblast orthochromatic, reticulocyte, erytrocyto, cells from a myeloid line, myeloblast, promyelocyte, myelocyte, metamyelocyte, neutrophil, eosinophil, basophilo, lymphocytic line, lymphoblast, prolymphocyte, lymphocyte, monocytic line, monoblast, promonocyte, monocyte, megakaryocyte, megakaryoblast platelets, promegakaryocyte platelets, megakaryocyte platelets, cells from a plasmatic line, B cell, plasmoblast, proplasmocyte, plasmocyte, hepatocytes, hystiocytes, microglia cells, fibroblasts, adipocytes, reticulocytes, chondrocytes, chondroblasts, osteocytes, osteoblasts, osteoclasts, cells with cilli, cells with flagellum, cells from a germinal cell line, cells from a ovogonia cell line, cells from a spermatogonian line, pneumocytes kind I and II, kidney cells, nephroblasts, retinocytes, retinoblasts, and oligodendrocytes.

Specific combinations determined to be useful for particular purposes are: pericyte cells and endothelial cells, which are useful to repair or create capillaries with applications or treatment of cardiovascular diseases and endothelial illness; hematopoietic cells and mesenchymal cells, which are useful to repair or create bone marrow with applications or treatment of stroma in leukemias, anemia, myeloproliferative diseases and thrombocythemia; hematopoietic cells and adipose cells, which are useful to repair or create bone marrow with applications or treatment of stroma in leukemias, anemia, myeloproliferative diseases and thrombocythemia; hematopoietic cells and bone cells, which are useful to repair or create bone marrow with applications or treatment of stroma in leukemias, anemia, myeloproliferative diseases and thrombocythemia; hematopoietic cells and fibroblast cells, which are useful to repair or create bone marrow with applications or treatment of stroma in leukemias, anemia, myeloproliferative diseases and thrombocythemia; fibroblast (basal) cells and glandular cells, which are useful to repair or create glands with applications or treatment of thyroid problems and diabetes; epithelial cells and glandular cells, which are useful to repair or create glands with applications or treatment of thyroid problems and diabetes; neural cells and oiligodendroglial cells, which are useful to repair or create nervous and neural tissues with applications or treatment of brain injury, stroke, spinal cord injury; embryonic stem cells and induced pluripotent stem cells, which are useful to repair or create mesenchymal stem cells (feeder) plus stem cells to repair or create embryonic and induced pluripotent stem cells clusters with applications or treatment employing in-vitro growth; fibroblast (feeder) cells and stem cells, which are useful to repair or create embryonic and induced pluripotent stem cells clusters with applications or treatment employing in-vitro growth; tumoral cells and hematopoietic cells, which are useful to create tumoral models with applications on drug screening and angiogenic applications; tumoral cells and endothelial cells, which are useful to create tumoral models with applications on drug screening and angiogenic applications; tumoral cells and fibroblast (stromal) cells, which are useful to create tumoral models with applications on drug screening and angiogenic applications; hepatocytes cells and stellate cells which are useful to repair or create liver tissues with applications or treatment of cirrhosis and hepatitis.

The above-named specific combinations of cells have been found to operate synergistically to enhance and improve cell assemblies on the surface of the bubble, gel or droplet that forms the reservoir. This surface is referred to as the interface or surface template. The synergistic effect also results in faster assembly of the artificial gland with a higher quality cellular shell or bio-membrane-layer coating the interface. Assembly time for these paired cells on the interface can be reduced by up to 50%. It has also been found that shell quality is much more homogeneous and uniform in terms of the distribution of cells. The quality of the array of cells on the interface has a direct effect on the survival of the cells as well as the membrane's mechanical properties, such as strength and permeability.

An alternative embodiment of the artificial gland uses the same configuration and components as described above, except that biological units are used instead of cells. The biological units form a membrane. The membrane is configured to define an enclosed micro-scale volume. A reservoir is within the enclosed micro-scale volume. The reservoir comprises a bio-reactor capable of containing a product of activity of the biological units. And, the reservoir comprises a substance selected from the group consisting of a gas, a liquid, and a gel. Biological units are similar in that they perform a biological activity that produces products, but they may not be classified as living. Biological units include fungi, algae, spores, pollen, yeast, bacteria, and viruses.

An alternative embodiment of the artificial gland uses the same configuration and components as described above, except that components of a cell are used instead of cells. The components of a cell form a membrane assembled in three dimensions. The membrane is configured to define an enclosed micro-scale volume. A reservoir is within the enclosed micro-scale volume. The reservoir comprises a bio-reactor capable of containing a product of activity of the components of a cell. The reservoir comprises a substance selected from the group consisting of a gas, a liquid, and a gel. Components of a cell are similar in that they perform a biological activity that produces products, but they are not classified as living. Examples of components of a cell are: enzymes, prions, hormones, growth factors, Tumor Necrosis Factor-alpha, Tumor Necrosis Factor-beta, cytokines, interleukins, albumin-scavengers, polyclonal-anti-bodies, monoclonal-anti-bodies, immunoglobulins, protease enzymes, lysosomes, vesicles, cell membranes, rough endoplasmic reticulums, smooth endoplasmic reticulums, mitochondria, ribosomic ribonucleic acid, transference ribonucleic acid, deoxyribonucleic acid, microtubules, endocrine cells, and human T-cells, fatty acids, beta-OH-butyrate, aceto acetate, polycations, poly L lysine, ornithine, chitosan, oligoelements, genes, chloroplasts, chlorophyll, glucidic elements.

Preferably, the reservoir in the artificial gland also includes nanoparticles, a buffer, a surfactant, and a gel precursor. These nanoparticles are biocompatible, tend to affix to the surface of the cells when in the aqueous solution, create a cation when exposed to an acid, and have physical and chemical characteristics that allow their removal from the cells without destroying all of the cells. The preferable nanoparticle is calcium carbonate that forms a +2 cation when exposed to acid. Magnesium carbonate is also known to be functional, and there may be many others.

The buffer is one that maintains a constant pH of the aqueous solution and many are known in the art. Preferable buffers are phosphate buffered saline (PBS) and Tris-buffered saline (TBS) containing 0.2% Tween-20 (TBST).

The surfactant is one that stabilizes droplets comprising the aqueous solution from coalescing upon contact and many are known in the art. Preferable surfactants are biocompatible surfactants for water-in-fluorocarbon emulsions synthesized by coupling oligomeric perfluorinated polyethers (PFPE) with polyethyleneglycol (PEG). To stabilize the drops, a PFPE-PEG block-copolymer surfactant is added to the suspending oil at a concentration of 1.8% (w/w)

The gel precursor that reacts with the cation to form a gel. For all the embodiments, preferred gel precursors are sodium alginate, calcium carbonate nanoparticles or calcium phosphate nanoparticles

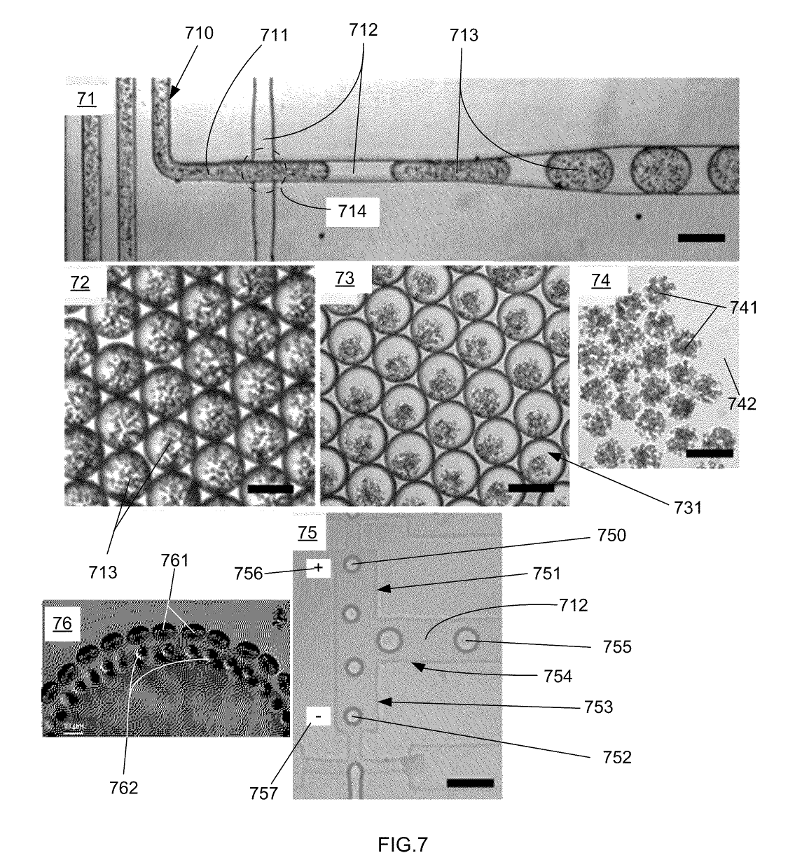

FIG. 7 provides micrographs that illustrate a first preferred method making an artificial gland (741). (Scale bar equals 100 micrometers). This first preferred method employs a droplet (713), electrocoalescence and controlled gelation. This first preferred method presents in a series of steps to create the droplet (713), combining them, forming artificial glands (741), and then isolating the created artificial glands.

The first preferred method first comprises a step of producing an aqueous solution (711). The aqueous solution (711) includes water, which is necessary for the solution to be aqueous. It further includes cells, nanoparticles, a buffer, a surfactant and a gel precursor. The aqueous solution may also include other components, such as for example, a hydrophobic dye, a hydrophilic dye, a protein, and a nutraceutical.

The nanoparticles are ones that are biocompatible, that tend to affix to the surface of the cells when in the aqueous solution, and that have physical and chemical characteristics that allow their removal from the cells without destroying all of the cells, preferably without destroying the vast majority of cells. Nanoparticles of calcium carbonate are preferred and nanoparticles of magnesium oxide are also known to meet these conditions.

The buffer maintains a constant pH of the aqueous solution and a variety of biocompatible buffers are well known in the art.

The surfactant stabilizes droplets made from the aqueous solution and retards their coalescing with each other upon contact. A variety of surfactants are well known in the art.

The gel precursor is a fluid that hardens in at slow enough rate to allow cells to migrate outside the gel at the interface of the gel and the water.

The first preferred method further includes a step of injecting the aqueous solution (711) in a microchannel (710). A microchannel is a micron sized pipe or pipette, typically found in a microfluidic device common in this field.

The first preferred method further includes a step of adding inert oil (712) to the first microchannel at an injection port (714). The inert oil (712) used in all embodiments of the invention is preferably one of the following: fluorocarbon oil, silicone oil, and/or fluorosilicone oil.

The step of adding inert oil (712) would be done at a point in the microchannel below or after the point where the aqueous solution is injected so that it has a chance to interrupt the flow of the aqueous solution and cause the aqueous solution to form droplets (713) within the inert oil (712) acting as a carrier fluid. Thus, the injection port (714) is configured so that the inert oil (712) separates the aqueous solution (711) into droplets (713), whereupon the droplets (713) are collected in a container (72). The inert oil (712) and surfactant maintain the droplets (713) in a discrete form and retard their recombination upon contact with each other.

The first preferred method next includes a step of adding acid to the container to reduce the pH of the droplets (713). Micrograph (73) shows the droplets (713) with acid having been added. The acid causes a reduction in pH within the droplets (713), which in turn causes the gel precursor to begin gelation (731). In order to enter the droplets (713) and start gelation (731), the acid must be miscible in the inert oil (712) and the droplets (713) of aqueous solution (711). When complete, gelation inside each droplet forms the artificial gland within each droplet. A slow gelation process enables the cells to migrate to the surface of the hardening gel within the droplets (713).

The first preferred method next includes a step of removing the inert oil (712) from the container (72). Micrograph 74 shows the artificial glands (741) within the droplets (713) after inert oil (712) removal. After the artificial gland is formed, the inert oil (712) must be drained or otherwise removed to isolate or separate the artificial glands (741) within the droplets (713) in the container (72) from the inert oil (712).

The first preferred method next includes a step of adding a salt (742) to the container to deactivate the surfactant and release each artificial gland from within its droplet. The salt counteracts the surfactant and destroys the integrity of the droplets (713). This causes the release of the artificial glands (741) from the droplets (713).

The first preferred method next includes a step of rinsing the artificial glands to remove the salt (742) and the deactivated surfactant from the container (72). Any additional components of the aqueous solution are also rinsed away in this step. FIG. 1 illustrates the resulting first artificial gland embodiment (100).

### Example 1

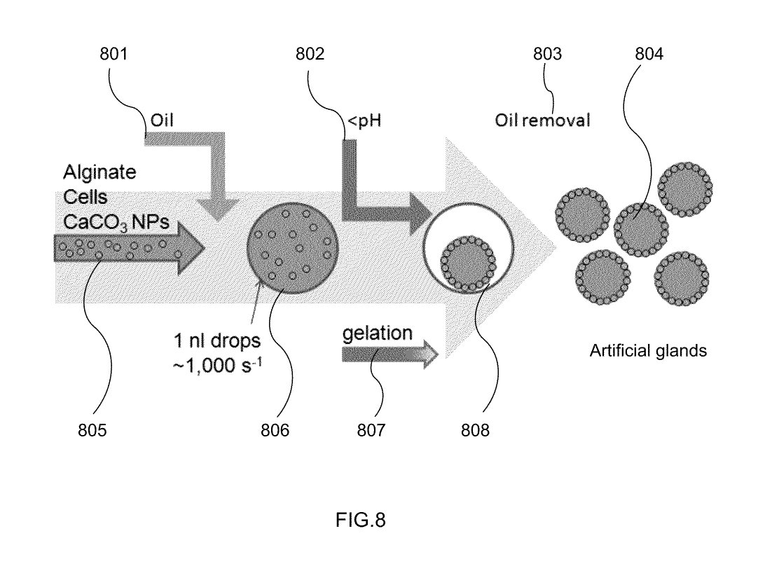

FIG. 8 illustrates a method of artificial gland production implemented as a proof of concept experiment using yeast (Saccharomyces cerevisiae) cells. Cells and a suspension of precipitated calcium carbonate nanoparticles (CaCO3 NPs) (805) with a 1% solution of sodium alginate in tris-buffered saline were mixed together (801). Precipitated calcium carbonate nanoparticles are not soluble at neutral pH, and therefore, the alginate remains in the liquid, uncrosslinked state within the drop (806).

The drop (805) size was 100 micrometers resulting in about 30 cells per drop (805). After drop (805) formation, its pH was reduced (802), which dissolves precipitated calcium carbonate nanoparticles and initiates crosslinking of sodium alginate. This was accomplished by flowing the drop into a second oil stream containing acetic acid, which partitions into the aqueous phase.

By observing the solidification in a flow chamber, it was noted that acid diffuses into the drops, generated the alginate gel (807) thus forming a phase within the aqueous drop (808). This process resulted in a high percentage of the cells presented on the surface of the gel phase (804).

The technique demonstrated with yeast cells is transferable to more complex cell types such as stem and mammalian somatic cells because cell droplet formation using this microfluidic system as well as alginate encapsulation has been conducted with a variety of mammalian somatic cells.

A second preferred method of making the first artificial gland embodiment (100) uses two droplets in a microfluidic device and combines them using electrocoalescence. The droplets may be produced in the same manner as described above for the first preferred method, differing in the components of the two droplets. FIG. 7 at micrograph (75) illustrates this method after the droplets are formed.

This second preferred method of making the artificial gland includes a step of producing a first droplet (750) in an inert oil (712) carrier fluid. The first droplet (750) comprises cells in a first aqueous medium. The first droplet (750) also includes a surfactant that stabilizes droplets made from the first aqueous medium and retards their coalescing upon contact with each other. The first droplet (750) may also include a buffer to maintain a constant pH in the first aqueous medium.

This second preferred method includes a step of producing a second droplet (752) in an inert oil (712) carrier fluid. The second droplet (752) comprises a second aqueous medium, calcium carbonate nanoparticles, a gel precursor, and a surfactant that stabilizes second droplets from coalescing with each other upon contact. The second droplet (752) may also include a buffer to maintain a constant pH in the first aqueous medium.

The second preferred method next a step of charging the first droplet (750) and the second droplet (752) with opposite electrical charges. Thus, if the first droplet (750) is positively charged (756), then the second droplet (752) is negatively charged (757). Alternatively, if the first droplet is negatively charged, then the second droplet (752) is positively charged.

The second preferred method next includes a step of combining the first droplet (750) with the second droplet (752) by colliding them together in a microchannel to produce a third droplet. FIG. 7 shows an upper microchannel (751) for the first droplet (750) and a lower microchannel (752) for the second droplet (752). The collision of the particles combined with the opposite electrical charge causes the droplets to combine into the third droplet (754).

The remaining steps in this second preferred method parallel those in the first preferred method after the step of adding acid to the container. Thus, these remaining steps are: collecting the third droplet (754) in a container; adding acid to the container to reduce the pH of the third droplet (754); removing the inert oil from the container; adding a salt to the container to deactivate the surfactant and release the artificial gland from within the third droplet (754); rinsing the artificial gland to remove the salt and the deactivated surfactant from the container. In this second preferred method, the acid is miscible in the inert oil carrier fluid, the first aqueous medium, and the second aqueous medium. Also, the acid initiates gelation inside each third droplet (754) and forms the artificial gland within each third droplet (754).

The electrocoalescence mechanism, which is involved in the second preferred method and other methods disclosed herein, is referred to as cross-linking and the artificial glands are cross linked either electrostatically or covalently. Coalescence is first induced by electrostatic attraction due to the opposite charges between the first droplet and the second droplet, or in other methods between the various artificial glands being combined. Electrocoalescence may also include subjecting the droplets, or artificial glands, being combined to an electric field, which has been shown to promote coalescence, that is, the merging process.

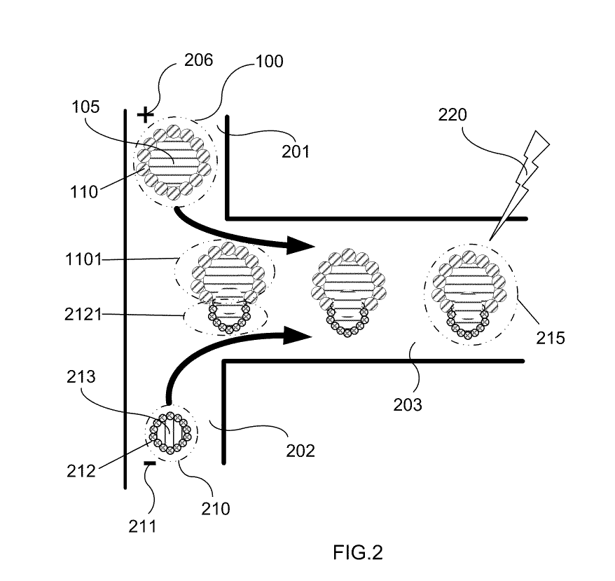

FIG. 2 illustrates a third preferred method of making a fourth artificial gland (215) with a plurality of types of cells in the membrane. It uses two artificial glands already created, one having a cell type in the membrane that is different from the other.

This third preferred method of making the artificial gland (215) includes a step of flowing, in a first microchannel (201), a first artificial gland embodiment (100) carrying an electric charge, in this case a positive electric (206). This first artificial gland embodiment (100) comprises a first reservoir (105) comprising a biocompatible liquid; and, a first membrane comprising a plurality of first cells (110) of the first artificial gland embodiment (100) surrounding the first reservoir (105).

This third preferred method next includes a step of flowing, in a second microchannel (202), a second artificial gland (210) carrying an electric charge opposite to that of the first artificial gland, in this case a negative electric charge (211). The second artificial gland (210) comprises a second reservoir (213) comprising a second biocompatible liquid. This may be the same biocompatible liquid as in the first reservoir (105), or it may be a different biocompatible liquid. The second artificial gland (210) includes a second membrane surrounding the second reservoir (213). The second membrane comprises second cells (212), that is, of a type different from the first cells (110) in the membrane of the first artificial gland embodiment (100).

This third preferred method next includes a step of contacting, or colliding, the first artificial gland embodiment (100) with the second artificial gland (210) upon their flowing to a junction connecting the first microchannel (201) and the second microchannel (202). The junction comprises a main microchannel (203). This structure is shown in FIG. 2 in graphic form and is identical to the structure shown in micrograph (75) in FIG. 7.

This third preferred method next includes a step of producing a third artificial gland (215) by merging the first artificial gland embodiment (100) and the second artificial gland (210) using electrocoalescence, as described above. This may be supplemented by other mechanisms (220) indicated by the electrical bolt in FIG. 2, to fix or stabilize the structure or freeze the structure of the third artificial gland, i.e., the Janus particle. These may include temperature treatment, exposure to light, or subjecting the third artificial gland (215) to an electric current.

The coalescence process is enhanced by the tendency of nano- and microparticles to assemble on liquid/liquid or liquid/gel interfaces. This tendency is thought to arise from the relatively large interfacial tension at the interface, such as an oil-water interface.

Electrocoalescence has been shown to result in controlled, non-spherical shapes as illustrated by the third artificial gland (215) in FIG. 2.

FIG. 2 shows the third artificial gland (215) comprises a membrane with a first discrete section (1101) comprising first cells (110) of the first artificial gland embodiment (100) and second discrete section (2121) comprising the cells (212) of the second artificial gland (210).

Optionally, the third preferred method includes a first artificial gland that further comprises a plurality of cells in the first reservoir. This is consistent with the third artificial gland embodiment (150) shown in FIG. 1.

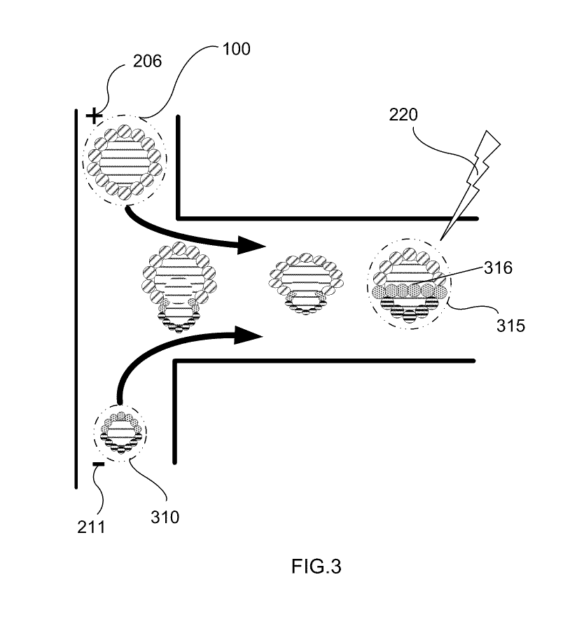

Optionally, the third preferred method includes a first artificial gland that further comprises a plurality of cell types in first membrane. This is consistent with the second artificial gland embodiment (125) shown in FIG. 1 and also illustrated in FIG. 3 for the complex artificial gland (310) wherein the two types of cells in the membrane are indicated by different shading or hatching.

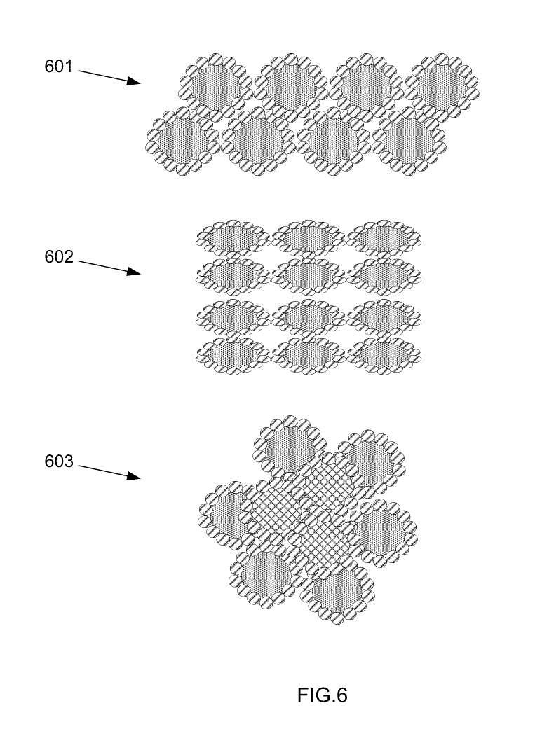

Optionally, the third preferred method further comprises the step of stacking a plurality of third artificial glands into a macroscopic network of close-packed arrays. Stacking is illustrated in FIG. 6 with three examples or motifs: a first motif (601); a second motif (602) and a third motif (603).

The first motif (601) is the stacking of the third artificial glands in close-packed arrays. First motif (601) is a starting point for creating tissue since cell proliferation and signaling will alter the original form.

Second motif (602) is a more directed organization where artificial glands are flattened and the elongated or disk-like artificial glands are layered as well as arrayed. In second motif (602), a denser network is formed and cell signaling can be better facilitated.

Third motif (603), spherical or spheroidal artificial glands are arranged in a spherical close-packed array. In third motif (603), artificial glands are packed in 3-dimensions based on cubic lattice, face-centered cubic lattice, and hexagonal lattice unit structures.

Using varied motifs, more complex structures can also be made by combining two or three of them. Also, the motifs are not limited to artificial glands and can be constructed from cellular subunits and colloidal nano or microparticles as well.

Optionally, the third preferred method further comprises the step of adding material to the macroscopic network. The material, for example, is any component that helps with biological activity. Preferably such material is a nutrient, a protein, a collagen, fibrinogen, elastin, a synthetic biocompatible polymer, a pharmaceutical product, a perfluorinated compound, and a biopolymer.

Application of the third preferred method can be used to create complex multiple membranes on and within the artificial gland. For example, the third artificial gland (215), which has a complex structure, may be similarly combined with another artificial gland to make a more complex fourth artificial gland.

### Example 2

FIG. 3 illustrates the merger of the first artificial gland embodiment (100) with a complex artificial gland (310) comprising a membrane with a plurality of cell types. The resulting artificial gland (315) comprises a complex membrane and a layer (316) within the reservoir.

### Example 3

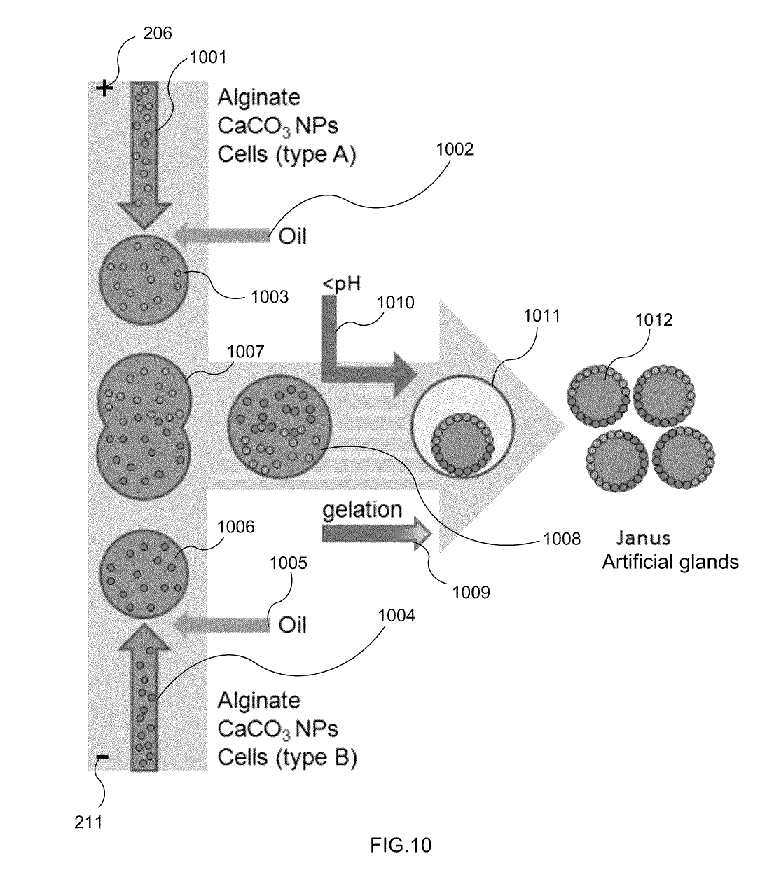

FIG. 10 illustrates another example implementing the invention to produce Janus artificial glands (1012). These are complex artificial glands typically having each hemisphere of the artificial gland comprising a different type of cell.

Two different artificial gland types (1001 and 1004) are encapsulated in drops (1003 and 1006) using oil (1002 and 1005) and flow in separate flow channels before meeting (1007) at a junction. The drops (1003 and 1006) are subjected to an electric field to promote merging into a unified drop (1008). In an electric field, drops attract each other and coalesce. Slow mixing in the microfluidic channels due to laminar flow and the internal flow patterns within the moving drops ensures that the different cell types will each remain in one hemisphere of the fused drop. The unified drop (1008) is subjected to gelation (1009) upon acidification (1010) to produce a hardened gel particle (1011).

### Example 4

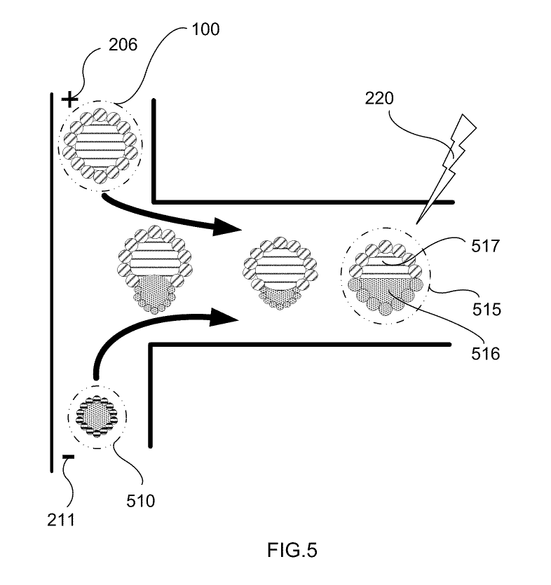

FIG. 5 is a flow diagram illustrating formation of an artificial gland with a variety of cells and liquids, also known as a double Janus structure. A first charged artificial gland embodiment (100) is combined with a fifth artificial gland (510) that is oppositely charged. The fifth artificial gland (510) comprises a plurality of cells in the membrane and a liquid core different from that of the first artificial gland embodiment (100). The merged particle (515) has a membrane comprising the two types of cells from the merged artificial glands and a core comprising a first liquid (516) distinct from a second liquid (517) liquids separated into two distinct regions.

Subsequent layers can be either homogenous, with new cell types or heterogeneous with similar or different cell types. Multiple applications of these steps can lead to sophisticated structures that grow in complexity and size to satisfy the complex developmental or treatment purposes.

For simple fluids or molecular surfactants, larger droplets that form after coalescence rapidly minimize their surface areas by forming a sphere. If, however, the colloidal particles are strongly bound to the interface, the relaxation of the droplet after coalescence jams the particles so that they form an elastic membrane. Further shape relaxation is prevented, and a dipolar shape can be frozen in place.

Particles whose radii exceed tens of nanometers are effectively irreversibly bound to the interface and can readily form spherical as well as non-spherical droplets and artificial glands.

The self-assembly of cells at the liquid/liquid interface is driven by the minimization of the interfacial energy and is enhanced by electrostatic interaction. The final artificial gland shape can be spherical, disk-like, or any other shape similar to the artificial glands produced in droplets or particles in microfluidic devices. By controlling the directionality of flow and hence the momentum of the artificial glands, coalescence can be tuned and orchestrated to form a highly complex membrane.

While a simple microchannel device is well known in the field, more complex microchannel networks can be easily envisioned to accommodate each increase in size and sophistication of design of the artificial gland.

A fourth preferred method of making the first artificial gland embodiment (100) mixes certain components together in a couple of steps, allowing droplets to form in the mixture, and then cleaning the mixture to isolate the artificial glands.

This fourth preferred method includes a step of preparing an aqueous culture medium comprising cells, polymers, and a protein composition. For all embodiments of the invention, preferred polymers are polylysine. A preferred protein composition is serum proteins.

This fourth preferred method next includes a step of injecting the aqueous culture medium into fluorinated oil. The fluorinated oil is preferably inert oil. Injection of the aqueous culture medium into fluorinated oil creates a suspension of discrete droplets of the aqueous culture medium.

This fourth preferred method next includes a step of forming a polymer monolayer on the surface of the droplet to form the artificial gland. The polymer monolayer automatically forms on the droplets in the suspension given the passage of time after the discrete droplets are formed, typically about 1-10 minutes after adding the components of the aqueous culture medium. The cells automatically migrate from within the droplet to the outside surface of the droplet given a sufficient residence time, typically about 1-10 minutes.

This fourth preferred method next includes a step of rinsing the artificial glands to remove the other suspension components and thereby producing isolated artificial glands. Once the polymer monolayer is formed, the artificial glands are formed and exist in the suspension.

A fifth preferred method of making the first artificial gland embodiment (100) is a variation of the fourth preferred method in that cells are injected after the polymer monolayer is formed on a droplet.

The fifth preferred method of making the first artificial gland embodiment (100) includes a step of preparing an aqueous culture medium comprising polymers, and a protein composition.

The fifth preferred method of making the first artificial gland embodiment (100) next includes a step of injecting the aqueous culture medium into fluorinated oil to form a suspension of discrete droplets of the aqueous culture medium.

The fifth preferred method of making the first artificial gland embodiment (100) next includes a step of forming a polymer monolayer on the surface of the droplet.

The fifth preferred method of making the first artificial gland embodiment (100) next includes a step of injecting cells into the suspension for assembly on the surface of the droplet to form the artificial gland.

The fifth preferred method of making the first artificial gland embodiment (100) next includes a step of rinsing the suspension to produce isolated artificial glands.

A sixth preferred method of making the first artificial gland embodiment (100) is a one that produces two types of droplets and then combines them in using microchannels to form the first artificial gland embodiment (100).

The sixth preferred method of making the first artificial gland embodiment (100) includes a step of preparing a first aqueous culture medium comprising polymers, and a protein composition.

The sixth preferred method of making the first artificial gland embodiment (100) includes a step of injecting the first aqueous culture medium into fluorinated oil to form a suspension of first droplets of the aqueous culture medium.

The sixth preferred method of making the first artificial gland embodiment (100) next includes a step of forming a polymer monolayer on the surface of the first droplets.

The sixth preferred method of making the first artificial gland embodiment (100) includes a step of producing second droplets in an inert oil carrier fluid. Each second droplet comprises cells in a second aqueous medium together with a surfactant. The surfactant stabilizes the second droplets and retards their coalescing upon contact with each other.

The sixth preferred method of making the first artificial gland embodiment (100) includes a step of charging the first droplets and the second droplets with opposite electrical charges;

The sixth preferred method of making the first artificial gland embodiment (100) includes a step of combining one of the first droplets with one of second droplets by colliding them together in a microchannel to produce a third droplet. Obviously, this is preferably done for a batch of droplets together, but is so worded to broaden the scope of the invention.

The sixth preferred method of making the first artificial gland embodiment (100) includes a step of collecting the third droplet in a container.

The sixth preferred method of making the first artificial gland embodiment (100) includes a step of adding acid to the container to reduce the pH of the third droplet. A preferred acid is acetic acid. The acid is one that is miscible in the inert oil carrier fluid, the first aqueous medium and the second aqueous medium. Miscibility is required because the acid must enter the droplet to effectuate a gelation process. The acid thus initiates gelation inside the third droplet and forms the artificial gland within the third droplet.

The sixth preferred method of making the first artificial gland embodiment (100) includes a step of removing the inert oil from the container.

The sixth preferred method of making the first artificial gland embodiment (100) includes a step of adding a salt to the container to deactivate the surfactant and release the artificial gland from within the third droplet.

The sixth preferred method of making the first artificial gland embodiment (100) includes a step of rinsing the artificial gland to remove the salt and the deactivated surfactant from the container.

A seventh preferred method of making the artificial gland is a one that first creates a droplet with a nanoparticle coating and then forms the artificial gland over that droplet. This droplet with the nanoparticle coating is constructed similarly to the cell coated nanoparticle (190) illustrated in FIG. 1, but instead of a cell (185) within the coating, it is a droplet.

The seventh preferred method of making the artificial gland includes a step of creating a suspension of nanoparticles in inert fluorocarbon oil.

The seventh preferred method of making the artificial gland next includes a step of flowing a fluid in a microchannel, wherein the fluid is selected from the group consisting of a gas, a liquid, and a gel. Preferred gases are air, carbon dioxide, or oxygen mixtures. Preferred liquids are aqueous solutions and a preferred gel comprises alginates.

The seventh preferred method of making the artificial gland includes a step of introducing the suspension into the microchannel to form a discrete volumetric packet of the fluid.

The seventh preferred method of making the artificial gland includes a step of producing a stabilized discrete volumetric packet comprising a layer of nanoparticles on the surface of the discrete volumetric fluid. The discrete volumetric packed formed in the previous step is stabilized by the layer or coating of nanoparticles.

The seventh preferred method of making the artificial gland includes a step of adding cells to the stabilized discrete volumetric packet so that the cells assemble in three dimensions and organize to form a membrane covering the discrete volumetric packet to produce the artificial gland.

An eighth preferred method of making the first artificial gland embodiment (100) uses droplets dispersed in oil with a surfactant in one microchannel to collide and electrocoalesce with cells from a second microchannel.

This eighth preferred method includes steps of: preparing individual aqueous droplets comprising water dispersed in inert oil and a surfactant; charging the individual aqueous droplets with an electric charge; flowing the aqueous droplets into a first microchannel; flowing cells carrying an electric charge opposite to the electric charge of the droplets into a second microchannel that intersects with the first microchannel; combining the droplets with the cells by colliding them together in a microchannel to produce a second droplet; collecting the second droplet in a container; adding acid to the container to reduce the pH of the second droplet, wherein the acid is miscible in the inert oil and the water, and wherein the acid initiates gelation inside each second droplet and forms the artificial gland within each second droplet; removing the inert oil from the container; adding a salt to the container to deactivate the surfactant and release the artificial gland from within the third droplet; and, rinsing the artificial gland to remove the salt and the deactivated surfactant from the container.

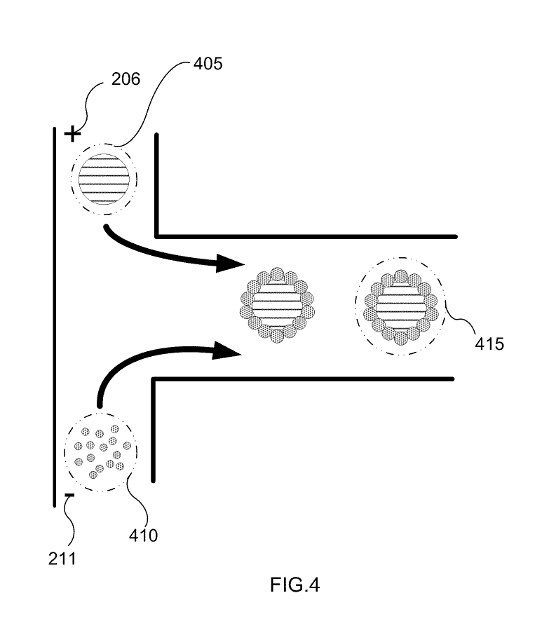

The eighth preferred method is illustrated in FIG. 4. It illustrates a microfluidic formation of an artificial gland by combining an individual aqueous droplet (405) dispersed in inert oil. The individual aqueous droplet (405) carries a positive electric charge (206) in one microchannel and the cells (410) carrying a negative electric charge (211) in another intersecting microchannel. The collision of the particles at the intersection creates the artificial gland (415).

In this eighth preferred method, each individual aqueous droplet (405) can function effectively as both a reaction vessel and a template for particle formation. The size and rate of droplet formation is controlled precisely through manipulation of the relative flow rates of the oil and aqueous phases and through modifications in the channel geometry. Typically, droplets are produced in the size range of 10-500 micrometers in diameter (about 1 picoliter to about 100 nanoliters in volume) at rates of up to 100,000 per second, which results in rapid formation of tens of millions of identical compartments.

The invention includes a precursor particle (190) illustrated in FIG. 1. The precursor particle (190) is used in the preparation of an artificial gland. The precursor particle (190) is composed of calcium carbonate nanoparticles (180) coating a cell (185). The nanoparticles form a protective coating over the cell (185). Thus, the precursor particle (190) comprises a coating of nanoparticles consisting essentially of calcium carbonate nanoparticles and a cell.

Similarly, FIG. 1 shows an embodiment of the invention comprising an artificial gland surrounded by nanoparticles (102). Alternative embodiment (101) in FIG. 1 uses, as an illustrative example, the first artificial gland embodiment (100) as the artificial gland surrounded by nanoparticles (102). However any artificial gland may be used.

The nanoparticles (102) form a second coating or membrane and protective covering over the artificial gland. The nanoparticles (102) are biocompatible, tend to affix to the surface of the cells when in an aqueous solution, create a cation when exposed to an acid, and have physical and chemical characteristics that allow their removal from the cells without destroying all of the cell. The nanoparticles are preferably made of calcium carbonate.

A ninth method of making an artificial gland (101) surrounded by nanoparticles (102) includes a step of combining cells and nanoparticles in water. The nanoparticles are of a biocompatible material that will migrate to the cells and homogenously surround each cell in the aqueous solution forming a membrane of nanoparticles. Biocompatibility essentially means that the nanoparticles are compatible with the cells such that while surrounding each cell, they preserve cell viability

The ninth method of making an artificial gland (101) surrounded by nanoparticles (102) next includes a step of removing the water to produce product cells each having a shell of nanoparticles;

The ninth method of making an artificial gland (101) surrounded by nanoparticles (102) next includes a step of adding an inert oil as a carrier fluid;

The ninth method of making an artificial gland (101) surrounded by nanoparticles (102) next includes a step of flowing the product cells and carrier fluid in a microchannel toward an intersecting microchannel.

The ninth method of making an artificial gland (101) surrounded by nanoparticles (102) next includes a step of flowing a discrete volumetric packet in a second microchannel toward the intersecting microchannel. This flow causes the volumetric packet to collide with the product cells and allows product cells to assemble and organize on the surface of the discrete volumetric packet. The discrete volumetric packet is a gas, a liquid, a gel, volvox algae or a combination of these.

Volvox algae, or simply volvox, is one of the best-known chlorophytes and is the most developed in a series of genera that form spherical colonies. Each mature volvox colony is composed of numerous flagellate cells similar to chlamydomonas, up to 50,000 in total, and embedded in the surface of a hollow sphere or coenobium containing an extracellular matrix made of a gelatinous glycoprotein. The cells swim in a coordinated fashion, with distinct anterior and posterior poles. The cells have eyespots, more developed near the anterior, which enable the colony to swim towards light. The individual algae in some species are interconnected by thin strands of cytoplasm, called protoplasmates.

Optionally, the ninth method of making an artificial gland (101) surrounded by nanoparticles (102) includes a step of adding a buffer to the water, cells and nanoparticles to maintain a constant pH of the combination.

Optionally, the ninth method of making an artificial gland (101) surrounded by nanoparticles (102) includes a step of charging the product cells and the discrete volumetric packet with opposite electrical charges.

FIG. 7 shows a micrograph (76) of a portion of an embodiment of the invention that comprises an artificial gland having a cellular membrane that coats a volvox algae colony (762) within the reservoir. The micrograph (76) is of red blood cells (761) forming a membrane coating a spherical volvox algae colony (762). This artificial gland is an independent micro-scale unit that promotes biological activity, which, in this case, the biological activity is partly in the algae that produces oxygen in the presence of light, thus, preserving the cells in the membrane. This invention includes cells assembled in three dimensions and organized to form a membrane, the membrane configured to define an enclosed micro-scale volume; and, a reservoir within the enclosed micro-scale volume, the reservoir comprising volvox algae.

A variation of this artificial gland is one where the volvox is replaced by other algae. It has the same components as described above, except that instead of volvox algae, the reservoir comprises an organized algae micro-colony. Preferably, this algae micro-colony is one or more of diatoms, cyanobacteria, pediastrum, hydrodictyon, chlorella, paramecium bursania, Haematococcus pluvialis, spirogyra, mougeotia and zygnema.

The unique biological organizational approach described herein can achieve laterally patterned functionality, induced coalescence of two particle-coated droplets, or phase separation of particles bound to a single droplet.

The method and design are amenable to the use of many kinds of materials (including organic or inorganic; edible; magnetic; etc.) and avoid the need for surfactants or scaffolds. They are also amenable to large-scale processing, thus providing the potential for low-cost artificial glands with highly tunable shape, elasticity, rheology, surface-adsorption, or other properties.

By extension, the methods described here is also unique for its flexibility and suitability for large-scale application. Oil-in-water or water-in-oil samples allow the encapsulation of hydrophobic or hydrophilic dyes, proteins, nutraceuticals, for example.

The artificial glands are composed of polymer or inorganic material. These polymer and inorganic material are in sizes in the range of nanometers to microns. For example, these can include PNIPAm microgel spheres, which expand or contract in response to heating, cooling, change of pH, or exposure to light of a specific wavelength. These spheres can endow the artificial glands with a triggerable response.

Use of paramagnetic, electrically conducting or insulating, and/or strongly scattering particles in assemblies of artificial glands can endow other physical properties that can be useful in the biological function of the artificial glands. The different type of cells or subcellular units can be combined in various arrangements is only limited for practical reasons rather than due to inherent limitations in the motif.

Other methods of making the artificial gland (101) involve double emulsions in a carrier fluid that move cells to a bubble or droplet surface. The carrier fluid is may be any biologically compatible hydrophobic liquid, or biologically compatible liquid that is poorly miscible with water, and that does not interfere with the emulsion process. This typically includes oils other than silicon oil. Preferred carrier fluids are fluorocarbon oil and/or fluorosilicone oil.

Each of these double emulsion methods includes steps of mixing silicon oil and sodium alginate to form a first emulsion having a pH of less than 7; mixing cells and calcium carbonate nanoparticles in water to form a second emulsion; mixing the first emulsion with the second emulsion a carrier fluid to form a double emulsion; and, mixing ABIL-EM 90 polymeric surfactant in the double emulsion.

The first emulsion forms small droplets in the carrier fluid that become surrounded by the second emulsion. The combination of these emulsions in the carrier fluid causes these small droplets to become surrounded by a shell of the second emulsion of water, cells and calcium carbonate. The shell forms a continuous inner boundary, or interface, between the two emulsions and a continuous outer boundary between the second emulsion and the carrier fluid. The last step of mixing ABIL-EM 90 polymeric surfactant in the double emulsion is one that aids formation of the artificial gland by repulsion of the cells from the continuous outer boundary towards the continuous inner boundary.

Repulsion of the cells from the continuous outer boundary towards the continuous inner boundary involves steric repulsion. A biocompatible ABIL-EM 90 polymeric surfactant is mixed in the emulsions. PPG (propargylglycine) groups stick into the continuous outer boundary, PEG (polyethyleneglycol) groups form a polymer brush extending outward from the continuous outer boundary into carrier fluid. The PEG polymer brush hinders protein absorption and cell adhesion at the continuous outer boundary.

Thus, each double emulsion method is enabled by employing two general strategies: repulsion of the cells from the continuous outer boundary towards the continuous inner boundary; and, attraction of the cells to the continuous inner boundary with five different methods.

The five different methods of attracting the cells to the continuous inner boundary involve (1) hydrophobic adhesion of integrin-receptor ligands; (2) polymerizating ECM at the continuous inner boundary; (3) using poly-NIPAM microgels in the second emulsion; (4) employing alginate polymerization at the continuous inner boundary; and, (5) enabling electrostatic adhesion of cells to the continuous inner boundary.

The first method of attracting the cells to the continuous inner boundary includes a step of adding cell growth medium, collagen and fibronectin monomers to the second emulsion. This step takes advantage of hydrophobic adhesion of integrin-receptor ligands. This method employs commonly used ligands, such as fibronectin and collagen, which adhere non-specifically to hydrophobic surfaces. The collagen and fibronectin monomers are small molecules that rapidly diffuse and adhere to the continuous inner boundary, to which the integrin-receptors on the cell surface will strongly bind.

The second method of attracting the cells to the continuous inner boundary includes steps of pre-emulsifying the silicon oil in an aqueous solution of thrombin; and, adding fibrinogen monomers to the second emulsion. This method is involves polymerization of an extracellular matrix of fibrin (ECM polymer) at the continuous inner boundary. This method employs integrin receptors that recognize and bind to the ECM polymer. A fibrin shell is created by pre-emulsifying silicon oil in an aqueous solution of thrombin, then adding fibrinogen monomers to the second emulsion. When added to the carrier fluid, the thrombin is driven to the continuous inner boundary by surface tension. The fibrinogen monomers polymerize into a fibrin network at the continuous inner boundary, catalyzed by the thrombin. Integrin receptors on cell surface naturally bind to the resulting network at the continuous inner boundary, i.e., the surface of the first emulsion droplet.

The third method of attracting the cells to the continuous inner boundary includes a step of adding poly-NIPAM (poly-N-isopropylacrylamide) microgels in the first emulsion. Cells naturally adhere to the thermo-responsive hydrogel, poly-NIPAM. Adhesion is enhanced by supplementing cell-growth medium with integrin-receptor binding ligands, collagen or fibronectin. At 37 degrees Centigrade, poly-NIPAM microgels are in a collapsed, hydrophobic state, and the small molecules fibronectin and collagen rapidly diffuse and adhere to the microgel surface. Cells readily adhere and spread onto the microgel surfaces.

The fourth method of attracting the cells to the continuous inner boundary includes steps of dissolving a small amount of sodium-acetate in the silicon oil of the first emulsion; and adding cell growth medium, sodium alginate and calcium carbonate nanocrystals, i.e. nanoparticles, to the second emulsion. This method employs alginate polymerization at the continuous inner boundary. The nanocrystals are locally dissolved when they come into contact with the sodium-acetate at the continuous inner boundary. Consequently, the alginate forms a thin shell of hydrogel at the continuous inner boundary, non-specifically trapping cells within the shell of hydrogel.

The fifth method of attracting the cells to the continuous inner boundary involves the steps of incubating the cells in growth medium supplemented with biocompatible cationic polymers; and, adding a biocompatible anionic surfactant to the first emulsion. This method takes advantage of electrostatic adhesion of cells to the continuous inner boundary. Preferred biocompatible cationic polymers include poly-L-lysine (PLL) or poly-diallyldimethylammonium chloride (PDAC). The polycations adsorb to the negatively charged cell surface. The biocompatible anionic surfactant promotes adhesion of surface modified cells to continuous inner boundary. A preferred biocompatible anionic surfactant is sodium laurylether sulphate.

Artificial glands having a non-spheroidal shape, such as the toroidal shape, can be designed and produced using a unique variation of the techniques and processes known for producing toroidal droplets using a single liquid, as described in “Generation and Stability of Toroidal Droplets in a Viscous Liquid” by E. Pairam and A. Fernandez-Nieves in PRL 102, 234501 (2009) PHYSICAL REVIEW LETTERS, 12 Jun. 2009, which is hereby incorporated by reference herein (herein referred to as “Pairam reference”).

In this unique variation of the Pairam reference, coaxial nozzles each containing an emulsion described above as the double emulsions are injected into a rotating bath which acts on the ejected emulsions to produce toroidal artificial glands. The specific components of each emulsion are as described above. For this process, the bath comprises a cell growth culture medium, such as Dulbecco's modified eagle medium (DMEM), which becomes the rotating bath and viscous carrier fluid. This method involves first continuously injecting each emulsion into the rotating bath through a metallic coaxial needle to form a dispersing coaxial liquid jet. The jet forms a tubular fiber with one emulsion on the outside and one on the inside. Stopping the continuous injection cuts off the fiber length. With the fiber present in the bath its ends comprising the special emulsions automatically join up to form a toroidal shape for the artificial gland.

Other shapes can also be made using this method, for example spherical double-emulsions by controlling the viscosity of the cell growth culture medium bath and several other variables described in the above noted literature for use with single liquids. Alternate shapes for the artificial gland rely on the viscous forces exerted by a rotating continuous cell growth medium over the coaxial liquid extruded from the coaxial injection needle. The resultant coaxial jet is forced to close into a torus due to the imposed rotation. Once formed, the torus can transform into single or multiple coaxial spheres.

Finally, it is noted that the artificial gland of the invention may be assembled using ink jet, also known as bio ink, printing processes.

### Example 5

In another example implementing the invention using the motifs as illustrated in FIG. 6 and described above, artificial glands are combined to form a macroscopic network creating 3-D organization useful for tissue engineering applications. In this example, nutrients, proteins, growth factors, chemical drugs, antibodies, ligands, etc. are encapsulated into the interior of the artificial glands to ensure survival/differentiation/proliferation/activation/structural changes of the cells as the 3D structure is being formed. Along with the nutrients etc, one can also employ materials mimicking the extra cellular matrix as components of the artificial gland. One such material is collagen. Other materials include fibrinogen, elastin and other biologically derived polymers or proteins that mimic the extracellular microenvironment. Either of these materials can be employed by themselves or in combination (as blends) with synthetic biocompatible or biodegradable polymers or biopolymers.

### Example 6

Another example of a method for assembling the artificial gland employs a modified ink jet printing, also known as bio ink, process. Commercially available inkjet printers have been successfully modified to specifically deliver artificial micro-glands units into scaffold fabricated according to a computer-aided design template. Examples: Hewlett Packard (HP) 550C computer printer and an HP 51626a ink cartridge or a Canon ink jet printer (Pixma ip4500) and ink cartridges (CLI, Y-M-C-BK, PGBK model) were reconstructed for micro-glands printing. Artificial micro-glands were suspended separately in a concentrated phosphate buffered saline solution. The independent micro-glands units were subsequently printed as a kind of “ink” onto several tiopapers made from soy agar and collagen gel. The control of developmental patterning through self-assembly involves physical mechanisms. Three-dimensional tissue structures are formed through the post-printing fusion of the micro-gland-ink particles. The computer-aided inkjet printing of viable independent micro-glands units holds potential for creating living tissue analogs, and may eventually lead to the construction of engineered human organs.

### Example 7

In another example implementing the invention, an artificial gland creates a realistic model to study tumors. Thus, one can introduce into the mixture artificial glands that have cancerous cells as membrane components. As the 3D structure comes together, one can carefully detect the spread of the cancerous cells in a realistic 3D model that is not constructed on a scaffold.

Besides the ability to obtain fundamental understanding of the spread of cancerous cells, one utility of the invention creating new strategies for limiting (or preventing) the spread of cancerous cells.

Another utility lies in the design of an artificial gland (3D structure) in order to study cell-cell and cell-microenvironment phenomena inside a tumor. Thus, given the ease of building such structures in accordance with the present invention, a vast number of models with different types of proteins and other drugs can be encapsulated to observe the effect on tumor cells.

### Example 8

In another example of the utility of the invention, a realistic 3D environment is created by sequential addition leading to an environment that can help in the study of stem cell behavior. For example, differentiation, pluripotency maintenance, growth capacity, etc. artificial glands can be used with embryonic or adult stem cells. One important factor is the size of the artificial gland. Since there is a fine level of control over the size and reproducibility of droplet formation in microfluidics, artificial glands offer a unique environment to both alter size and observe different effects on stem cell differentiation.

Because embryonic stem cells, including induced pluripotent stem cells, sometimes require a feeder layer for growing, this can be achieved with the present invention in two ways: by using stem cells to form the membrane of the artificial gland and by encapsulating the stem cells in the artificial gland. Alternatively, one can have different types of cells in the core and in the membrane.

In addition to size, the invention has utility in using small molecules, proteins, growth factors, etc. to control differentiation in a 3D model using artificial glands. Other factors can also be varied such as the presence of proteins, physical constraints, etc. in order to change the environment of the artificial gland for the study of pluripotency maintenance, differentiation, genetic stability, etc.

### Example 9

In another example of the utility of the invention, the invention's scaffold-free 3D structure provides a realistic model to aid drug development. Additives to the mixture that leads to the formation of artificial glands may consist of different drugs or pharmaceutical products. Also, various cell types can be added (including adult and embryonic stem cells as well as differentiated cells) so as to closely resemble the body.

Drugs may also be added during culture, after artificial glands formation. As the 3D structure comes together, one can detect drug effects on the growth and proliferation of cells in a 3D scaffold-free environment. This would also be a model for detecting the effect of drugs on pregnant women and the fetus. Besides the ability to obtain fundamental understanding one may also develop the upper limits of drug dosage in order to avoid unwanted side effects.

### Example 10

In another example of the utility of the invention, artificial glands may be used to build glands for cell therapy and gene therapy. Cell therapy has emerged as one of the most promising approaches to treat or potentially cure a number of diseases and disorders related to the central nervous system (e.g. Parkinson's, Alzheimer, Huntington), endocrine system (e.g. diabetes), heart disease, kidney failure, cancer, etc.

Cell microencapsulation has shown considerable promise in cell therapy since it offers better immunoprotection when donated cells are employed. Nevertheless, the materials employed in the encapsulation process often result in an inflammatory response and loss of cell viability in the early stages. For treating diabetes, one can build artificial glands incorporating islet cells in such a way that they would be essentially immuno-compatible.

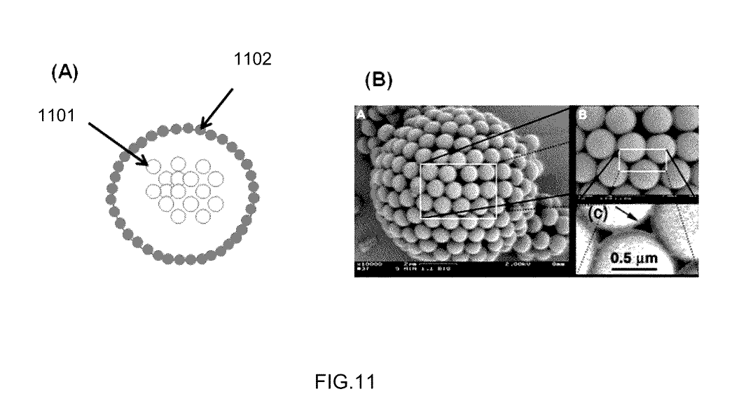

The artificial gland design shown in FIG. 11 consists of a membrane/reservoir structure in which islet cells are contained within the reservoir while the surrounding membrane consists of living cells that are “invisible” to the host immune system. The surrounding membrane consists of living cells that have been derived from a patient (directly differentiated cells or by using induced pluripotent stem (iPS) cell technology to obtain autologous iPS cells and differentiate them to the desirable cellular phenotype).

Any other cell that has immunotolerant properties may also be employed for the membrane. The host cells/tissue that come in contact with the living cells on the surface of the artificial glands, which are preferably derived from the patient, will only induce a negligible degree of inflammation. This type of artificial gland can be obtained either by electrocoalescence (Janus particles) of two different artificial glands or using just one type of artificial gland. In either case, the choice of material is critical so that the cells useful for cell therapy remain in the core and do not migrate to the surface due to lower interfacial energy.

One way to accomplish this utility is by physical means. By increasing the viscosity of the liquid core in order to achieve a gel-like consistency, migration of the cells (useful for cell therapy) will be limited and hence so will the inflammatory response. Alternatively, one can also employ chemical means such as functionalization of the living cells (autologous) for preferential migration to the interface leaving the cells useful for cell therapy within the core.

To ensure the survival of the cell relevant to cell therapy, nutrients can be included in the reservoir along with cells. These nutrients include growth factors, proteins and oxygen providing materials such as perfluorinated compounds and any other materials that maintain the viability of the encapsulated cells.

In addition to nutrients, the choice of materials can also impact the survival of the cells in the reservoir. Thus one can employ alginates, agarose or other polymers that have been employed in the literature or natural polymers such as collagen, hyaluronic acid, etc., either by themselves or in conjunction with other biocompatible polymers.

Additionally, an appropriate choice of the living cells can help long-term survival of cells within the reservoir.

### Example 11

Another utility of the invention is the use of stem cells (adult, embryonic, induced pluripotent stem, etc.) in the membrane, preferably stem cells that are derived from the patient. One such example is the use of mesenchymal stem cells as the peripheral cells since they are known as immunomodulators in maintenance of transplantation tolerance and auto-immunity.

Specifically, it has been hypothesized that the non-immunogenicity of mesenchymal stem cells is a consequence of: (a) lack of expression of major histocompatibility complex (MHC) (class II) molecules (before differentiation) on the surface; (b) ability to suppress T lymphocyte activation and proliferations due to a lack of ligands for CD28 and other co-stimulatory molecules on their plasma membrane; and, (c) establishment of chimerism through thymic and extrathymic deletion of autoreactive T-cell clones that down regulate effectors of T-cell responses.

### Example 12

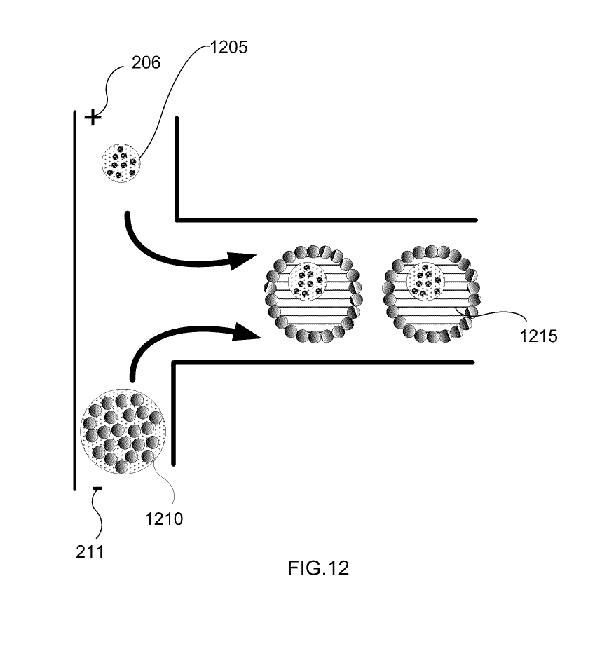

In another example of the utility of the invention, the invention may be used for the creation of artificial glands with immune invisible cells, such as mesenchymal stem cells on the surface of the droplet and encapsulated islets in the core, and is based on fusing drops within microfluidic channels using electric fields, as illustrated in FIG. 12. FIG. 12 is a flow diagram illustrating formation of artificial gland (1215) for insulin delivery/release applications combining two artificial glands (1205 and 1210) according to the invention.

### Example 13

Another example of the utility of the invention is for the treatment of type 1 diabetes. The human pancreas consists of about 10 billion beta cells where these cells at the end of their life cycle are constantly replaced by new beta cells generated in the pancreas. In type 1 diabetes this replacement is severely compromised due to autoimmune attack, which results in a dramatic depletion of beta cells.

While type 1 diabetes is normally treated by exogenous insulin therapy, a preferred alternative therapy is beta cell replacement or transplantation of islets of Langerhans. However, several barriers must be overcome before this procedure evolves from the current experimental stage to clinical use. The most common problem is that of host rejection.

To circumvent host rejection and avoid the deleterious side effects of immunosuppressive regimens, immunoisolation, techniques such as macro- or microencapsulation in alginate gels, agarose gels, biomaterial membranes, etc. have been tried.

While microencapsulation appears to show promise by offering better immunoprotection, the material employed in the encapsulation process often result in an inflammatory response. This can result in loss of viability of the islet cells in the early stages.

To improve islet viability, artificial glands that have immuno-compatible properties can be employed. The design, consistent with that shown in FIGS. 11 and 12, consists of a membrane-reservoir structure with mesenchymal stem cells on the surface of the droplet and encapsulated islets in the interior islet cells. Mesenchymal stem cells are employed as the peripheral cells given their role as immunomodulators in maintenance of transplantation tolerance and auto-immunity. Thus, encapsulated islet cells in artificial glands will result in a negligible inflammatory response for the treatment of diseases, thereby reducing cell necrosis without diminishing the efficacy of cell therapy.

### Example 14

Other examples of the utility of the invention are in the treatment of several forms of lung disease. The lung is the essential respiration organ in air-breathing animals. Its principal function is to transport oxygen from the atmosphere into the bloodstream and to release carbon dioxide from the bloodstream into the atmosphere. This exchange of gases is accomplished in the mosaic of specialized cells that form millions of tiny, exceptionally thin-walled air sacs called alveoli. Lung diseases include asthma, chronic obstructive pulmonary disease (COPD), for example and an especially devastating disease involving the lung is Cystic Fibrosis (CF).

### Example 15

Although technically a rare disease, cystic fibrosis is ranked as one of the most widespread life-shortening genetic diseases. It is most common among nations in the Western world where one in twenty-two people of Mediterranean descent is a carrier of one gene for CF, making it the most common genetic disease in these populations. In the United States, 1 in 4,000 children are born with CF. In 1997, about 1 in 3,300 caucasian children in the United States was born with cystic fibrosis.

CF is caused by a mutation in the gene cystic fibrosis transmembrane conductance regulator (CFTR). The product of this gene is a chloride ion channel important in creating sweat, digestive juices and mucus. Although most people without CF have two working copies (alleles) of the CFTR gene, only one is needed to prevent cystic fibrosis. CF develops when neither allele can produce a functional CFTR protein. Therefore, CF is considered an autosomal recessive disease.

The protein created by this gene is anchored to the outer membrane of cells and acts as a channel connecting the inner part of the cell (cytoplasm) to the surrounding fluid. This channel is primarily responsible for controlling the movement of chloride from inside to outside of the cell; however, in the sweat ducts it facilitates the movement of chloride from the sweat into the cytoplasm. When the CFTR protein does not work, chloride is trapped inside the cells in the airway and outside in the skin. Because chloride is negatively charged, positively charged ions cross into the cell because they are affected by the electrical attraction of the chloride ions. Sodium is the most common ion in the extracellular space and the combination of sodium and chloride creates the salt, which is lost in high amounts in the sweat of individuals with CF. This lost salt forms the basis for the sweat test.

How this malfunction of cells in cystic fibrosis causes the clinical manifestations of CF is not well understood. One theory suggests that the lack of chloride exodus through the CFTR protein leads to the accumulation of more viscous, nutrient-rich mucus in the lungs that allows bacteria to hide from the body's immune system. Another theory proposes that the CFTR protein failure leads to a paradoxical increase in sodium and chloride uptake, which, by leading to increased water reabsorption, creates dehydrated and thick mucus. Yet another theory focuses on abnormal chloride movement out of the cell, which also leads to dehydration of mucus, pancreatic secretions, biliary secretions, etc. These theories all support the observation that the majority of the damage in CF is due to blockage of the narrow passages of affected organs with thickened secretions. These blockages lead to remodeling and infection in the lung, damage by accumulated digestive enzymes in the pancreas, blockage of the intestines by thick feces, etc.

Several mechanical techniques are used to dislodge sputum and encourage its expectoration. In the hospital setting, chest physiotherapy is utilized. In addition, newer methods such as Biphasic Cuirass Ventilation, an associated clearance mode available in such devices, now integrate a cough assistance phase, as well as a vibration phase for dislodging secretions. Biphasic Cuirass Ventilation is also shown to provide a bridge to transplantation and are both portable and adaptable for home use. Aerosolized medications that help loosen secretions include dornase alfa and hypertonic saline. As lung disease worsens, breathing support from machines may become necessary. Individuals with CF may need to wear special masks at night that help push air into their lungs. During severe illness, people with CF may need to have a tube placed in their throats and their breathing supported by a ventilator.

### Example 16

Lung transplantation often becomes necessary for individuals with cystic fibrosis as lung function and exercise tolerance declines. Although single lung transplantation is possible in other diseases, individuals with CF must have both lungs replaced because the remaining lung would contain bacteria that could infect the transplanted lung. A pancreatic or liver transplant may be performed at the same time in order to alleviate liver disease and/or diabetes.

Gene therapy holds promise as a potential avenue to cure cystic fibrosis. Gene therapy attempts to place a normal copy of the CFTR gene into affected cells. Studies have shown that to prevent the lung manifestations of cystic fibrosis, only 5-10% of the normal amount of CFTR gene expression is needed. Multiple approaches have been tested for gene transfer, such as liposomes and viral vectors in animal models and clinical trials. However, at this time gene therapy is still a relatively inefficient treatment option. Ideally, transferring the normal CFTR gene into the affected epithelium cells would result in the production of functional CFTR in all target cells, without adverse reactions or an inflammation response. But if too few cells take up the vector and express the gene, the treatment has little effect. Additionally, problems have been noted in cDNA recombination, such that the gene introduced by the treatment is rendered unusable.

### Example 17

Recent literature suggests that adult bone marrow-derived cells can localize to the lung and acquire immunophenotypic characteristics of lung epithelial cells. It is speculated this might be a potential therapeutic approach for correcting defective lung epithelium in cystic fibrosis.

Mesenchymal stem cells are a population of stem cells in bone marrow. Recent reports suggest that mesenchymal stem cells can also differentiate into non-stromal tissues, including lung epithelial cells. These data provide a strong rationale to explore the potential use of mesenchymal stem cells for the treatment of lung diseases.

Mesenchymal stem cells have several appealing properties including the fact that they are readily isolated from patients by simple bone marrow aspiration and expand in culture. However, the cells are not immortal or tumorigenic. They can be readily transduced with viral or nonviral vectors for gene correction.

Gene-corrected stem cells can be infused back to the same patient to achieve autologous treatment, thus avoiding the problem of immune rejection and the need for immune suppression. One possible approach is the use of allogenic mesenchymal stem cells, without a gene correction, in order to take advantage of their immunomodulation properties. Another option is to use allogenic MSC plus an immunosuppression regimen, in a similar manner to lung transplantation. These appealing features make mesenchymal stem cells good candidates for potential therapeutic applications.

In prior art systemic administration of adult stem cells from bone marrow in mice after total-body irradiation showed that the administered stem cells were mainly engrafted in alveolar spaces and sometimes in conducting airway-reported engraftment rates of adult bone marrow stem cell derived cells ranged from 0% up to 20% in the lungs, and from 0.025% up to 4% in conducting airway. Despite this low engraftment level, there is evidence that transplanted stem cell post-irradiation have some therapeutic effects. Alternatively, considering the advantages of the intratracheal route to target the airway and the respiratory epithelium, more recent studies reported the intratracheal administration of adult stem cells in reagent-injured lungs.

### Example 18

The intratracheal administration of mesenchymal stem cells in artificial glands could offer additional utility over individual cells.

The current literature indicates that adult stem cells from the lung are able to form multicellular spheroids (e.g., bronchospheres). Bronchospheres are composed of cells with a high expression of stem cell regulatory genes, which are not or only weakly detectable in the tissue of their origin. Morphological analysis showed that bronchospheres are composed of mixed phenotype cells with type II alveolar and Clara cell features, highlighting their airway resident cell origin. In addition to displaying specific pulmonary and epithelial commitment, bronchospheres showed mesenchymal features.

### Example 19

A utility of the present invention lies in using mesenchymal stem cells in artificial glands that behave as bronchospheres, improving their stem cell-like qualities, specifically their ability to differentiate into pneumocytes.

### Example 20

Another utility of the present invention lies in delivery of artificial glands in an aerosol. In this way, administration to the patient would be greatly facilitated.

The generation of artificial glands that imitate alveoli or bronchiole would generate an in vitro 3D model to study this therapy (and other applications such as drug testing, etc). Thus, another utility of the present invention lies in artificial glands that can be created to imitate alveoli and/or bronchioles. Artificial glands would be created directly from lung cells and/or with mesenchymal stem cells that are subsequently differentiated into pneumocytes.

Moreover, with this kind of celloidomes, in vitro, the effect of drugs, growth factors, hormones, or other compounds Placed within the artificial glands (imitating an airway administration into the lung) and/or outside of artificial glands (imitating a systemic administration) can be studied.

Another attractive option is the creation of artificial glands containing mesenchymal stem cells in the core. This multi-artificial gland would imitate in vitro the effect of mesenchymal stem cells intratracheal administration, and would permit the study of the ability of mesenchymal stem cells to integrate and/or differentiate into pneumocytes and/or muscle cells.

### Example 21

Another utility of the present invention lies in skin replacement. In the current state of the art, artificial skin is constructed from human keratinocytes and dermal fibroblasts grown from neonatal foreskin cultured on a matrix of type I collagen and has layers of cells similar to human skin, but lacks sweat glands and hair follicles.

A fibroblast is a type of cell that synthesizes the extracellular matrix and collagen, the structural framework (stroma) for animal tissues, and plays a critical role in wound healing. Fibroblasts are the most common cells of connective tissue in animals. Fibroblasts are morphologically heterogeneous with diverse appearances depending on their location and activity. Though morphologically inconspicuous, ectopically transplanted fibroblasts can often retain positional memory of the location and tissue context where they had previously resided, at least over a few generations. An artificial gland constructed with a fibroblast membrane has been constructed for testing the invention.

During the development of a hair follicle, there a group of dermal cells initiate the complex structure that will ultimately create a hair. Artificial glands can be formed from dermal cells to imitate the primordial hair follicle which, when introduced into artificial skin, would generate hair.

In another in vivo embodiment, artificial glands can be implanted directed under the skin of people who have lost their hair through a naturally process or due to an accident.

This type of technology could be expanded to create additional skin cell structures such as arrector pili muscle, sebaceous gland, sweat glands, melanocytes, etc.

The above-described embodiments including the drawings are examples of the invention and merely provide illustrations of the invention. Other embodiments will be obvious to those skilled in the art. Thus, the scope of the invention is determined by the appended claims and their legal equivalents rather than by the examples given.

### INDUSTRIAL APPLICABILITY

The invention has application to the biological and biomedical applications industry.

What is claimed is:

1. An artificial gland that is an independent unit for promoting biological activity, the artificial gland comprising:
cells assembled in three dimensions and organized to form a membrane, the membrane configured to define an enclosed volume; and,
a reservoir within the enclosed volume, the reservoir comprising a bio-reactor capable of containing a product of activity of the cells.

2. The artificial gland of claim 1, wherein the bio-reactor comprises a substance selected from the group consisting of a gas, a liquid, and a gel.

3. The artificial gland of claim 1, wherein the artificial gland has a dimension not exceeding 500 microns.

4. The artificial gland of claim 1, wherein the cells are selected from the group consisting of stem cells, mesenchymal cells, embryonic cells, hybridomes B, hybridomes T, differentiated cells, tumor cells, cancer cells, skin cells, neural tube cell derivatives, astrocytes, olygodendrocytes, neuron, muscle cells, myocytes, myocardiocytes, leiomyocytes, epithelial cells, endothelium cells, endocrine gland cells, immune system cells, phagocytes, macrophages, lymphocytes, white cells, thrombocytes, platelets, erythrocytes, red cells, neutrophils, mastocytes, eosinophils, hematopoietic precursor cells, cells from a erytocyte line, proerytroblast, erythroblast basophil, erythroblast polychromatophilo, erythroblast orthochromatic, reticulocyte, erytrocyto, cells from a myeloid line, myeloblast, promyelocyte, myelocyte, metamyelocyte, neutrophil, eosinophil, basophilo, lymphocitic line, lymphoblast, prolymphocyte, lymphocyte, monocytic line, monoblast, promonocyte, monocyte, megakaryocyte, megakaryoblast platelets, promegakaryocyte platelets, megakaryocyte platelets, cells from a plasmatic line, B cell, plasmoblast, proplasmocyte, plasmocyte, hepatocytes, hystiocytes, microglia cells, fibroblasts, adipocytes, reticulocytes, chondrocytes, chondroblasts, osteocytes, osteoblasts, osteoclasts, cells with cilli, cells with flagellum, cells from a germinal cell line, cells from a ovogonia cell line, cells from a spermatogonian line, pneumocytes kind I and II, kidney cells, nephroblasts, retinocytes, retinoblasts, and oligodendrocytes.

5. The artificial gland of claim 1, wherein the reservoir is further comprising:
nanoparticles that are biocompatible, tend to affix to the surface of the cells when in the aqueous solution, create a cation when exposed to an acid, and have physical and chemical characteristics that allow their removal from the cells without destroying all of the cells;
a buffer that maintains a constant pH of the aqueous solution;
a surfactant that stabilizes droplets comprising the aqueous solution from coalescing upon contact; and,
a gel precursor that reacts with the cation to form a gel.

6. A method of making the artificial gland of claim 5 using a droplet and controlled gelation, the method comprising the steps of:
producing an aqueous solution comprising:
cells;
nanoparticles that are biocompatible, tend to affix to the surface of the cells when in the aqueous solution, create a cation when exposed to an acid, and have physical and chemical characteristics that allow their removal from the cells without destroying all of the cells;
a buffer that maintains a constant pH of the aqueous solution;
a buffer that maintains a constant pH of the aqueous solution;
a surfactant that stabilizes droplets comprising the aqueous solution from coalescing upon contact; and,
a gel precursor that reacts with the cation to form a gel;
injecting the aqueous solution in a microchannel;
adding inert oil to the microchannel at an injection port, wherein the injection port is configured so that the oil separates the aqueous solution into droplets;
collecting the droplets in a container;
adding acid to the container to reduce the pH of the droplets, wherein the acid is miscible in the inert oil and the droplets, and wherein the acid initiates gelation inside each droplet and forms the artificial gland within each droplet;
removing the inert oil from the container;
adding a salt to the container to deactivate the surfactant and release each artificial gland from within its droplet; and,
rinsing the artificial glands to remove the salt and the deactivated surfactant from the container.

7. The artificial gland of claim 1, wherein the reservoir is further comprising cells.

8. The artificial gland of claim 1, further comprising nanoparticles surrounding the artificial gland to form a second membrane and protective covering over the artificial gland wherein the nanoparticles are biocompatible, tend to affix to the surface of the cells when in an aqueous solution, create a cation when exposed to an acid, and have physical and chemical characteristics that allow their removal from the cells without destroying all of the cell.

9. A method of making the artificial gland of claim 8, the method comprising the steps of:
combining cells and nanoparticles in water wherein the nanoparticles are configured to:
migrate to the cells and homogenously surround each cell in the aqueous solution forming a membrane of nanoparticles;
be compatible with the cells such that while surrounding each cell preserves their viability;
removing the water to produce product cells each having a shell of nanoparticles;
adding an inert oil as a carrier fluid;
flowing the product cells and carrier fluid in a microchannel toward an intersecting microchannel;
flowing a discrete volumetric packet in a second microchannel toward the intersecting microchannel so as to collide with the product cells and allow product cells to assemble and organize on the surface of the discrete volumetric packet, wherein the discrete volumetric packet is the selected from the group consisting of a gas, a liquid, a gel, and volvox algae.

10. The method of claim 9, further comprising the step of adding a buffer to the water, cells and nanoparticles to maintain a constant pH of the combination.

11. The method of claim 9, further comprising the step of charging the product cells and the discrete volumetric packet with opposite electrical charges.

12. A method of using the artificial gland of claim 1 comprising the step of depositing a plurality of artificial glands on a template comprising collagen, procollagen, elastin, fibronectin, laminin, alginate, alginate and polycations shell (poly L lysine, ornithine, chitosan, peg, poli metilen co guanidine, poly etilen amine, poteroglycans, heparin-sulfate, chondroitin-sulfate, keratin sulfate, polyacrylates, polyglicocolic acid, polyglicocolic acid and lactic acid, K-carrageenan, agarose, damaged tissues, artificial bone, and artificial muscle.

13. A method of making the artificial gland of claim 1 using two droplets with one of the droplets comprising cells, the method comprising the steps of:
producing a first droplet in an inert oil carrier fluid, the first droplet comprising cells in a first aqueous medium and a surfactant that stabilizes first droplets from coalescing with each other upon contact;
producing a second droplet in an inert oil carrier fluid, the second droplet comprising a second aqueous medium, calcium carbonate nanoparticles, a gel precursor, and a surfactant that stabilizes second droplets from coalescing with each other upon contact;
wherein the first droplet or the second droplet further comprises a buffer that maintains a constant pH of the first aqueous medium or the second aqueous medium, respectively;
charging the first droplet and the second droplet with opposite electrical charges;
combining the first droplet with the second droplet by colliding them together in a microchannel to produce a third droplet;
collecting the third droplet in a container;
adding acid to the container to reduce the pH of the third droplet, wherein the acid is miscible in the inert oil carrier fluid and the first aqueous medium and the second aqueous medium, and wherein the acid initiates gelation inside each third droplet and forms the artificial gland within each third droplet;
removing the inert oil from the container;
adding a salt to the container to deactivate the surfactant and release the artificial gland from within the third droplet; and,
rinsing the artificial gland to remove the salt and the deactivated surfactant from the container.

14. A method of making artificial gland of claim 1, the method employing a plurality of types of cells in the membrane, the method comprising the steps of:
flowing, in a first microchannel, a first artificial gland carrying an electric charge, the first artificial gland comprising:
a first reservoir comprising a biocompatible liquid; and,
a first membrane comprising a plurality of cells of a first type surrounding the first reservoir;
flowing, in a second microchannel, a second artificial gland carrying an electric charge opposite to that of the first artificial gland, the second artificial gland comprising:
a second reservoir comprising a second biocompatible liquid; and,
a second membrane surrounding the second reservoir wherein the second membrane comprises cells of a second type;
contacting the first artificial gland with the second artificial gland upon their flowing to a junction connecting the first microchannel and the second microchannel, said junction comprising a main microchannel; and,
producing a third artificial gland by merging the first artificial gland and the second artificial gland using electrocoalescence, wherein the third artificial gland comprises a membrane with a first discrete section comprising the cells of the first type and a second discrete section comprising the cells of the second type.

15. The method of claim 14, wherein the first reservoir further comprises a plurality of cells.

16. The method of claim 14, wherein the first membrane further comprises a plurality of cell types.

17. The method of claim 14, further comprising the step of stacking a plurality of third artificial glands into a macroscopic network of close-packed arrays.

18. The method of claim 17, further comprising the step of adding material to the macroscopic network, said material selected from the group consisting of a nutrient, a protein, a collagen, fibrinogen, elastin, a synthetic biocompatible polymer, a pharmaceutical product, a perfluorinated compound, and a biopolymer.

19. The method of claim 14, wherein using electrocoalescence comprises subjecting the first artificial gland and the second artificial gland to an electric field.

20. A method of making the artificial gland of claim 1 comprising the steps of:
preparing an aqueous culture medium comprising cells, polymers, and a protein composition;
injecting the aqueous culture medium into fluorinated oil to form a suspension of discrete droplets of the aqueous culture medium;
forming a polymer monolayer on the surface of the droplet to form the artificial gland; and,
rinsing the suspension to produce isolated artificial glands.

21. A method of making the artificial gland of claim 1 comprising the steps of:
preparing an aqueous culture medium comprising polymers, and a protein composition;
injecting the aqueous culture medium into fluorinated oil to form a suspension of discrete droplets of the aqueous culture medium;
forming a polymer monolayer on the surface of the droplet;
injecting cells into the suspension for assembly on the surface of the droplet to form the artificial gland; and,
rinsing the suspension to produce isolated artificial glands.

22. A method of making the artificial gland of claim 1 comprising the steps of:
preparing a first aqueous culture medium comprising polymers, and a protein composition;
injecting the first aqueous culture medium into fluorinated oil to form a suspension of first droplets of the aqueous culture medium;
forming a polymer monolayer on the surface of the first droplets;
producing second droplets in an inert oil carrier fluid, the second droplet comprising cells in a second aqueous medium and a surfactant that stabilizes droplets comprising the second aqueous solution from coalescing upon contact;
charging the first droplets and the second droplets with opposite electrical charges;
combining one of the first droplets with one of second droplets by colliding them together in a microchannel to produce a third droplet;
collecting the third droplet in a container;
adding acid to the container to reduce the pH of the third droplet,
wherein the acid is miscible in the inert oil carrier fluid, the first aqueous medium and the second aqueous medium; and,
wherein the acid initiates gelation inside the third droplet and forms the artificial gland within the third droplet;
removing the inert oil from the container;
adding a salt to the container to deactivate the surfactant and release the artificial gland from within the third droplet; and,
rinsing the artificial gland to remove the salt and the deactivated surfactant from the container.

23. A method of making the artificial gland of claim 1 comprising the steps of:
creating a suspension of nanoparticles in an inert fluorocarbon oil;
flowing a fluid in a microchannel, wherein the fluid is selected from the group consisting of a gas, a liquid, and a gel;
introducing the suspension into the microchannel to form a discrete volumetric packet of the fluid;
producing a stabilized discrete volumetric packet comprising a layer of nanoparticles on the surface of the discrete volumetric fluid; and,
adding cells to the stabilized discrete volumetric packet so that the cells assemble in three dimensions and organize to form a membrane covering the discrete volumetric packet to produce the artificial gland.

24. A method of making the artificial gland of claim 1 comprising the steps of:
preparing individual aqueous droplets comprising water dispersed in inert oil and a surfactant;
charging the individual aqueous droplets with an electric charge;
flowing the aqueous droplets into a first microchannel;
flowing cells carrying an electric charge opposite to the electric charge of the droplets into a second microchannel that intersects with the first microchannel;
combining the droplets with the cells by colliding them together in a microchannel to produce a second droplet;
collecting the second droplet in a container;
adding acid to the container to reduce the pH of the second droplet,
wherein the acid is miscible in the inert oil and the water, and,
wherein the acid initiates gelation inside each second droplet and forms the artificial gland within each second droplet;
removing the inert oil from the container;
adding a salt to the container to deactivate the surfactant and release the artificial gland from within the third droplet; and,
rinsing the artificial gland to remove the salt and the deactivated surfactant from the container.

25. A method of making the artificial gland of claim 1 using a double emulsion, the method comprising the steps of:
mixing silicon oil and sodium alginate to form a first emulsion having a pH of less than 7;
mixing cells and calcium carbonate nanoparticles in water to form a second emulsion;
mixing the first emulsion with the second emulsion a carrier fluid to form a double emulsion; and,
mixing ABIL-EM 90 polymeric surfactant in the double emulsion.

26. The method of claim 25, further comprising the step of adding cell growth medium, collagen and fibronectin monomers to the second emulsion.

27. The method of claim 25, further comprising the steps of:
pre-emulsifying the silicon oil in an aqueous solution of thrombin; and,
adding fibrinogen monomers to the second emulsion.

28. The method of claim 25, further comprising the step of adding poly-NIPAM microgels in the first emulsion.

29. The method of claim 25, further comprising the steps of:
dissolving a small amount of sodium-acetate in the silicon oil of the first emulsion; and,
adding cell growth medium, sodium alginate and calcium carbonate nanocrystals to the second emulsion.

30. The method of claim 25, further comprising the steps of:
incubating the cells in growth medium supplemented with biocompatible cationic polymers; and,
adding a biocompatible anionic surfactant to the first emulsion.

31. An artificial gland of micro-scale for promoting biological activity, the artificial gland comprising:
biological units assembled in three dimensions and organized to form a membrane, the membrane configured to define an enclosed micro-scale volume; and,
a reservoir within the enclosed micro-scale volume, the reservoir comprising a bio-reactor capable of containing a product of activity of the biological units, wherein the reservoir comprises a substance selected from the group consisting of a gas, a liquid, and a gel.

32. The artificial gland of claim 31 wherein the biological units are selected from the group consisting of: fungi, algae, spores, pollen, yeast, bacteria, and viruses.

33. An artificial gland of micro-scale for promoting biological activity, the artificial gland comprising:
components of a cell assembled in three dimensions and organized to form a membrane, the membrane configured to define an enclosed micro-scale volume; and,
a reservoir within the enclosed micro-scale volume, the reservoir comprising a bio-reactor capable of containing a product of activity of the components of a cell, wherein the reservoir comprises a substance selected from the group consisting of a gas, a liquid, and a gel.

34. The artificial gland of claim 33 wherein the components of a cell are selected from the group consisting of: enzymes, prions, hormones, growth factors, Tumor Necrosis Factor-alpha, Tumor Necrosis Factor-beta, cytokines, interleukins, albumin-scavengers, polyclonal-anti-bodies, monoclonal-anti-bodies, immunoglobulins, protease enzymes, lysosomes, vesicles, cell membranes, rough endoplasmic reticulums, smooth endoplasmic reticulums, mitochondria, ribosomic ribonucleic acid, transference ribonucleic acid, deoxyribonucleic acid, microtubules, endocrine cells, and human T-cells, fatty acids, beta-OH-butyrate, aceto acetate, polycations, poly L lysine, ornithine, chitosan, oligoelements, genes, chloroplasts, chlorophyll, glucidic elements.

35. An artificial gland that is an independent micro-scale unit for promoting biological activity, the artificial gland comprising:
cells assembled in three dimensions and organized to form a membrane, the membrane configured to define an enclosed micro-scale volume; and,
a reservoir within the enclosed micro-scale volume, the reservoir comprising volvox algae.

36. An artificial gland that is an independent micro-scale unit for promoting biological activity, the artificial gland comprising:
cells assembled in three dimensions and organized to form a membrane, the membrane configured to define an enclosed micro-scale volume; and,
a reservoir within the enclosed micro-scale volume, the reservoir comprising an organized algae micro-colony selected from the group consisting of diatoms, cyanobacteria, pediastrum, hydrodictyon, chlorella, paramecium bursania, Haematococcus pluvialis, spirogyra, mougeotia and zygnema.
